(19) United States
(12) Patent Application Publication
(10) Pub. No.: US 2025/0277189 A1
(43) Pub. Date: Sep. 4, 2025

(54) METHOD FOR PRODUCING LEATHER USING CELL CULTURE ON HONEYCOMB MACROPOROUS POLYMERIC SCAFFOLD

(71) Applicant: FAIRCRAFT, Evry-Courcouronnes (FR)

(72) Inventors: Cesar VALENCIA GALLARDO, Saint Mande (FR); Elias AL TAWIL, Evry-Courcouronnes (FR); Sergiy KYRYACHENKO, Sartrouville (FR); HaÏkel BALTI, Paris (FR)

(21) Appl. No.: 18/857,952

(22) PCT Filed: Apr. 21, 2023
(86) PCT No.: PCT/EP2023/060468

§ 371 Date: Oct. 18, 2024

(30) Foreign Application Priority Data
Apr. 21, 2022 (EP) 22305597.1

(51) Int. Cl. C12N 5/077; D06N 3/00; D06N 3/12

(52) U.S. Cl. CPC C12N 5/0656; D06N 3/0043; D06N 3/123; C12N 2513/00; C12N 2533/40; C12N 2533/54

(57) ABSTRACT

The present disclosure relates to a method for producing leather comprising the step of culturing cells on honeycomb macroporous polymeric scaffold. The present disclosure also relates to leather obtainable by this method.

### TECHNICAL FIELD

The present disclosure relates to a method for producing leather comprising the step of culturing cells on honeycomb macroporous polymeric scaffold. The present disclosure also relates to leather obtainable by this method.

### BACKGROUND OF THE INVENTION

Leather was ranked the first most resource intensive material used in the fashion industry, far ahead of cotton or polyester. While animal leather is appreciated for its durability and aesthetics, it carries multiple environmental challenges: intensive natural resources use, animal welfare and working conditions concerns. Moreover, for many applications, leather needs to be exempt from defaults such as scratches or insect bites. As traditional leather production relies largely on cattle farming, up to 80% of hides are discarded due unmet quality standards.

As brands are under tremendous pressure to become more ethical and sustainable, a sustainable alternative to animal leather based on cell biology is needed.

Although synthetic leather was developed to address some of these concerns, it lacks the quality of natural leather. Previous attempts to make engineered leathers have been described. For example, EP1589098 describes a method of growing fibroblasts seeded onto three-dimensional bioactive scaffolds. The scaffolds may be made from collagen waste material from a tanning process (“split”), microparticles of pure collagen, particles of collagen waste material, or synthetic scaffolds (e.g., made of polymers such as HYAFF). WO2017/184967 describes the culture of stem cells or keratinocytes on collagen or PET membranes with micropores of 4 μm. However, these culture conditions are not optimal for cell growth and collagen production.

The tanning procedure breaks down most of the cellular, molecular and extracellular matrix components. The only components that resist storage and tanning are the collagen fibers and elastin fibers that are found in the leather in its final state and are secreted by fibroblasts (Sharphouse, J. H. Leather Technician's Handbook. Leather Producer's Association. p. 104. ISBN 0-9502285-1-6). These collagen and elastin fibers are responsible for the mechanical properties of leather. For this reason, inducing the secretion of Extracellular Matrix (collagen, elastin, among others) by the network of fibroblasts proliferating into scaffolds is a key step in the development of leather.

Thus, there is still a need to develop cell culture methods that promote cell proliferation as well as the production of collagen essential for high-quality leather production.

### SUMMARY OF THE INVENTION

The inventors developed a new method for producing high-quality leather by culturing fibroblasts in vitro on a honeycomb macroporous polymeric scaffold that allows efficient high cell attachment, cell culture and proliferation for long periods of time with minimal stress and damage for cells and results in a larger production of collagen by the fibroblasts in order to produce a collagen-rich extracellular matrix to be tanned and turned into a leather like biofabricated material.

In particular, the inventors produced a scaffold with a homogeneous but irregular distribution of honeycomb pores that gives rise to a well-organized structure with a homogeneous distance between the pores. Interestingly, this scaffold allows to maintain cell viability accompanied by a high secretion of extracellular matrix in comparison to other sponge macroporous scaffolds.

Honeycomb architecture of the pores opened on the surface and aligned finger glove-shaped pores progressing in the deep of the membrane scaffold according to the disclosure in addition to the interconnected pores direct multicellular organization and increase fibrillar collagen deposition. The well-organized membrane scaffold added to the stiffness provides a suitable microenvironment for cell colonization and development and the deposition of extracellular matrix (ECM) fibers.

This honeycomb macroporous scaffold, also referred herein as membrane scaffold is particularly useful to obtain a tissue particularly adapted to give a leather-like crafted product after tanning step.

The present invention relates to a method for producing leather comprising the steps of: a) culturing fibroblasts in vitro on a honeycomb macroporous polymeric scaffold to obtain a tissue wherein a surface of said scaffold comprises macropores of diameter comprised between 100 and 280 μm, more preferably between 110 and 225 μm with a pore area distribution at the surface comprised between 40% to 95%, preferably between 50% and 95%, 55% and 95%, 60% and 95%, 70% and 95%, more preferably 75 and 95% and wherein the pore wall thickness is below 70 μm, preferably below 60 μm, more preferably comprised between 2 and 50 μm, b) tanning said tissue obtained in step a) thereby forming said leather. In a preferred embodiment, said scaffold comprises polyester, preferably biodegradable and/or biosourced polyester, more preferably at a concentration comprised between 1 and 50% (w/w), preferably between 5 and 20% (w/w). In a particular embodiment, said macropores are interconnected with micropores, preferably said micropores having a diameter of less than 20 μm, preferably less than 10 μm. According to the present disclosure, preferably said scaffold has a tensile strength comprised between 0.1 and 10 MPa, 0.1 and 8 Mpa, 0.1 and 5 Mpa, 0.1 and 2 Mpa, 0.1 and 1 Mpa, preferably between 0.1 and 0.8 MPa, more preferably 0.2 and 0.7 MPa, and/or a Young's modulus comprised 5 and 1 MPa, preferably 5 and 800 kPa, 5 and 700 kPa, 5 and 600 kPa, 5 and 500 kPa, 5 and 400 kPa, 5 and 300 kPa, 5 and 200 kPa, preferably 5 and 100 kPa, 10 and 80 kPa, 40 and 70 kPa, 20 and 60 kPa, more preferably 30 and 50 kPa. In a preferred embodiment, said scaffold is obtained by non-solvent induced phase separation processes (NIPS).

In a particular embodiment, said scaffold is an asymmetric scaffold comprising macropores that are homogeneously but not regularly distributed over the whole thickness of the scaffold, in particular which presents a first face comprising macropores opened at the surface of diameter comprised between 100 and 280 μm, preferably between 110 and 225 μm with a surface pore area distribution comprised between 40% to 95%, preferably between 50% and 95%, 55% and 95%, 60% and 95%, 70% and 95%, more preferably 75 and 95% and pore wall thickness below 70 μm, preferably below 60 μm, more preferably comprised between 2 and 50 μm and a second opposite face comprising only nanopores of diameter below 10 nm, preferably 5 nm. In another particular embodiment, said scaffold is a symmetric scaffold comprising macropores which presents two faces comprising macropores of diameter comprised between 100 and 280 μm, preferably between 110 and 225 μm with a surface pore area distribution comprised between 40% to 95%, preferably between 50% and 95%, 55% and 95%, 60% and 95%, 70% and 95%, more preferably 75 and 95% and pore wall thickness below 70 μm, preferably below 60 μm, more preferably comprised between 2 and 50 μm.

In a preferred embodiment, a bioactive molecule is grafted at the surface of said scaffold, preferably said bioactive molecule is collagen, glucides, or glucides derived molecules such as glycosaminoglycan.

In another aspect, the present disclosure relates to a tissue obtainable by the culture step a) of the method of the present invention and a tanned leather obtainable by the method as described above.

Finally, the present disclosure relates to the use of a macroporous polymeric scaffold for producing leather, preferably by tanning tissue obtained from fibroblastic cell culture on said macroporous scaffold, wherein a surface of said scaffold comprises macropores of diameter comprised between 100 and 280 μm, preferably between 110 and 225 μm with a surface pore area distribution comprised between 40% to 95%, preferably between 50% and 95%, 55% and 95%, 60% and 95%, 70% and 95%, more preferably 75 and 95% and pore wall thickness below 70 μm, preferably below 60 μm, more preferably comprised between 2 and 50 μm.

### LEGEND FIGURES

FIG. 1: Scheme of the polyester asymmetric membrane scaffold production method by NIPS processes.

FIG. 2: Scheme of the production of a symmetric membrane scaffold by physical and/or chemical methods for removing the asymmetrical scaffold smooth face.

FIG. 3: Scanning electron microscope images of porous face (a), the interior of the macropores (b), the transversal slice (c), and the smooth face (d) of a biodegradable polyester membrane scaffold. Porous face (a) showing the presence of honeycomb-like macropores on top of the porous face, the micropores connecting the macropores (b), low-thickness smooth face on the other side of the porous face, and the absence of macro and microporous on the smooth face. Scale bars: 400 μm (a, c and d) and 200 μm (b).

FIG. 4: Scanning electron microscopy of polyester sponges seeded with fibroblast cells, fixed and dried. Cell bodies lining the entire surface of the polyester sponge scaffold. Scale bars: 400 μm (a and b) and 50 μm (c).

FIG. 5: Scanning electron microscopy of polyester membranes seeded with fibroblast cells. Cells colonizing the scaffold with abundant extracellular matrix fibers deposition. Scale bars: 400 μm (a and b) and 50 μm (c).

FIG. 6: Proliferation of fibroblasts cultured into polyester sponge (round, full line) and membrane (squared, pointed line) scaffolds at different time points.

FIG. 7: Tissue production by fibroblastic cells seeded into polyester membrane scaffolds after 1 (a) and 3 (b) weeks of seeding and observed by fluorescence microscopy. Collagen secretion from cells was observed by fluorescence microscopy using an anti-collagen I antibody (Sigma, ref. C2456). Cell nuclei were stained with DAPI (blue) and collagen I (green). Scale bar: 500 μm.

FIG. 8: Tanning process with tanning/re-tanning processes of tissue samples to obtain a tanned leather a) Tanning and retanning process were carried into a tanning drum, b) Surface image of tanned leather made by light microscopy, scale bar: 2 mm. c) Final result of the obtained tanned leather.

FIG. 9: Schematic replication (right) of the borders of opened pores at the surface of polyester sponge (up) and membrane (down) scaffolds based on Scanning electron microscopy images (left).

FIG. 10: Surface porosity percentage (%) distribution of the macropores on the surface of sponges (gray) and membrane (black) scaffolds. The median±MAD % surface porosity of membrane and sponge scaffolds is respectively 66.11±8.53 and 21.49±8.65. Wilcoxon sum-rank test p-value=0.0011655, ** p<0.01.

FIG. 11: Pore wall thickness between the opening macropores of sponge (gray) and membrane (black) scaffolds obtained by calculation of the distance between two pore centers diminished by the sum of the radius. The median±MAD wall thickness of membrane and sponge scaffolds is respectively 29.37±12.40 and 70.15±26.60. Wilcoxon sum-rank test p-value=, **** p<0.0001.

FIG. 12: Representative result of the traction mechanical tests performed on polyester scaffolds following the ISO 3376:2020 standard using an Instron 34SC-1 system equipped with a 50N load cell (CAT. NO.: 2530-50N) and manual tightening grips (CAT. NO.: 2710-203). a) Recorded stress/strain traction curve of a polyester membrane scaffold, b) Determination of the elastic limit of the sample to measure the Young's modulus, and c) Determination of the tear load of the sample. The speed is imposed during the test and set at 100 mm/min. Strength and displacement measurements are recorded. Stress is calculated with the initial thickness and width of the sample. Elongation is calculated with the initial length and the observed displacement.

FIG. 13: Directionality of biomatter fibers (cells and extracellular matrix) in the surface of in vitro skins. Directional field lines of biomatter in the surface of in vitro skins obtained by scanning electron microscopy (A and B), Scale bar: 100 μm. Directionality histogram showing the frequency of the biomatter directional fields of a surface of in vitro skin samples (C).

FIG. 14: Thermograms of the DSC analyses of the samples: macroporous scaffold (FSC), in-vitro skin (FP), leather of in-vitro skin tanned with aldehyde (CF-1).

FIG. 15: Decomposition of the peaks and the determination of the denaturation temperatures of the different species present in the sample: Onset temperature (Tₒₙₛₑₜ) of the beginning of the transition, denaturation temperature corresponding to the peak (Td) and offset temperature of the end of the transition (T_{offset}).

### DETAILED DESCRIPTION OF THE INVENTION

The inventors have developed a method for producing high-quality leather using an in vitro culture step of fibroblasts on a honeycomb macroporous polymeric scaffold to improve cell growth and collagen secretion which provides the stiffness, mechanical strength and resistance to abrasion found in leather. In particular, the inventors produced a honeycomb macroporous polymeric scaffold with a homogeneous distribution of pores at the surface of said scaffold that gives rise to a well-organized structure with a homogeneous distance between the pores.

Interestingly, this scaffold allows to maintain cell viability accompanied by a high secretion of extracellular matrix in comparison to sponge macroporous scaffolds. The high secretion of extracellular matrix of the cell cultures on this scaffold allows it to produce a leather of high-quality.

The present disclosure relates to a method for producing leather comprising the steps of:
a) culturing fibroblasts in vitro on a honeycomb macroporous polymeric scaffold to obtain a tissue wherein a surface of said scaffold comprises macropores of diameter comprised between 100 and 280 μm, preferably between 110 and 225 μm with a surface pore area distribution comprised between 40% to 95%, preferably between 50% and 95%, 55% and 95%, 60% and 95%, 70% and 95%, more preferably 75 and 95%,
b) tanning said tissue thereby forming said leather.

According to the present disclosure, the term “scaffold” refers to a tridimensional support which provides physical and structural support for cells and allowing tissue formation.

According to the method of the present disclosure, the surface of said scaffold comprises macropores of diameter comprised between 100 and 280 μm, preferably between 110 and 225 μm in which the cells can penetrate, thus conferring a biological environment favoring the proliferation and the function of the fibroblasts.

For the purposes of the present disclosure, by the term “pore diameter” is intended the diameter average mean of the pore as measured with Scanning Electron Microscopy (SEM) by averaging the diameter of 5 to 30 pores measured using the ImageJ software (Rasband, W. S., ImageJ, U. S. National Institutes of Health, Bethesda, Maryland, USA, https://imagej.nih.gov/ij/, 1997-2018). In a preferred embodiment, said pore diameter is measured at one of the surfaces of the scaffold.

To maintain the mechanical structure of the scaffold while providing a suitable biological environment for cell proliferation, the surface of honeycomb macroporous scaffold according to the present disclosure has a surface pore area distribution comprised between 40% to 95%, preferably between 50% and 95%, 55% and 95%, 60% and 95%, 70% and 95%, more preferably 75 and 95%.

Surface pore area distribution at the surface of the scaffold can be determined by analyzing representative images of the microarchitecture of the scaffold obtained by Scanning Electron Microscopy (SEM). Surface pore area distribution refers to the ratio of pore surface area to the overall scaffold surface area. The surface porosity (surface pore area distribution) of fabricated membranes was calculated by the following equation:

$$P o {r}_{s u f} = {\int }_{0}^{h} \frac{( {A}_{P} ) z * d z}{{A}_{t}}$$

where: Aₜ is the total area of image, Aₚ is the area porosity at distance z, h is height of image. The surface pore area distribution corresponds to the percentage occupied by pores as identified by the electron microscopy image at a selected magnification. The images can be analyzed using appropriate software that are able to measure the dimension of pores sections and surface pore area distribution visible on the scaffold surface. The surface area porosity can be determined as described in the example D. 2. of the present application.

According to a particular embodiment, the macroporous scaffold preferably has a degree of total porosity varying from 65% to 98%, in particular from 70 to 98%, 75 to 98%, more preferably 80% to 95%.

The “degree of porosity”, “void fraction” or “total porosity” is different from surface pore area distribution as described above. Indeed, for the purposes of the present invention, by the term “degree of porosity” or “void fraction” or “total porosity” is intended to mean the percentage of the volume of the pores relative to the volume of the material, as measured according to a gravimetric method (Guarino V, et al. 2008 September; 29(27):3662-70). The volume of the sample was calculated from the measurements of the sample dimensions. Measurement of the mass of the sample allowed to deduce the sample density. For each type of sample, the density (ρSc) was the average value obtained from 4 samples. Porosity of the scaffold was then calculated using the formulae:

$$Porosity = ( 1 - ( ρ Sc / ρ Ppoly ) ) : 100$$

where ρPpoly is the density of the polymer according to the manufacturer's database.

The scaffold according to the present disclosure is a honeycomb macroporous scaffold (also called herein membrane scaffold) that is a well-organized structure with a homogeneous distance between the pores, i.e., the walls separating the pores have the same thickness.

In a preferred embodiment, on the surface of the honeycomb macroporous scaffold according to the present disclosure, the distance between pores that corresponds to pore wall width is below 70 μm, preferably below 60 μm, more preferably comprised between 2 and 50 μm.

As used herein, pore wall width refers to the distance between two opposite walls of the pore. Pore wall width can be measured by any methods known in the art. In a particular embodiment, the pore wall width can be measured from SEM images for example by calculating the distance between pores as described in Haeri, Morteza & Haeri, Mohammad. (2015); Journal of Open Research Software; 3. 10.5334/jors.bn and in the example D.2. of the present application. The distance between pores corresponds to the average surface area between the pores that can be calculated from the calculation of the distance between the centers of two neighboring pores diminished by the sum of the radius of each pore. In another embodiment, the pore wall width can also be calculated by the modeling of the geometry of the surface of the scaffold as described in example D.2. of the present application. This method consists of the modeling of the geometry of the surface and transpose the pore surface area into a circle with a mean radius determined by the average of the distance of pixelated points on the line from the center of this area.

The honeycomb macroporous scaffold according to the present disclosure can be engineered to present a thickness that is adapted for the leather synthesis. In particular, the scaffold can have a thickness from 0.1 to 2.5 mm and in particular from 0.3 to 2 mm.

The thickness of the sample can be measured following the ISO 2589:2016 standard method. Thickness is measured with a digital thickness gauge J-40-L manufactured by Checkline that complies with ISO 2589:2016. Thirty measurements were made, distributed over the sample and averaged.

In a particular embodiment, the scaffold according to the present disclosure has a tensile strength comprised between 0.1 and 10 MPa, 0.1 and 8 Mpa, 0.1 and 5 Mpa, 0.1 and 2 Mpa, 0.1 and 1 Mpa, preferably between 0.1 and 0.8 MPa, more preferably 0.2 and 0.7 MPa.

In another particular embodiment, the scaffold according to the present disclosure has a Young's modulus comprised between 5 and 1 MPa, preferably 5 and 800 kPa, 5 and 700 kPa, 5 and 600 kPa, 5 and 500 kPa, 5 and 400 kPa, 5 and 300 kPa, 5 and 200 kPa, preferably 5 and 100 kPa, 10 and 80 kPa, 40 and 70 kPa, 20 and 60 kPa, more preferably 30 and 50 kPa.

Physical and mechanical tests on the scaffolds, said the determination of tensile strength and Young's modulus were performed following the ISO 3376:2020 standard. Environment conditions are ambient temperature and humidity. Instron 34SC-1 system is used to perform the tests. It is equipped with an Instron 50N load cell (CAT. NO.: 2530-50N) and Instron manual tightening grip (CAT. NO.: 2710-203). The speed is imposed during the test and set at 100 mm/min. Strength and displacement measurements are recorded. Stress applied during the test is calculated by the following equation:

$$Stress = \frac{F}{{S}_{T S}} = \frac{F}{W * {T}_{T S}}$$

where: F is traction force applied by the system over the surface of the tensile specimen; S_{TS} is the minimal surface of the tensile specimen, and W and T_{TS} are the width and the thickness of the tensile specimen.

Elongation is calculated with the initial length and displacement and displacement on the traction axis.

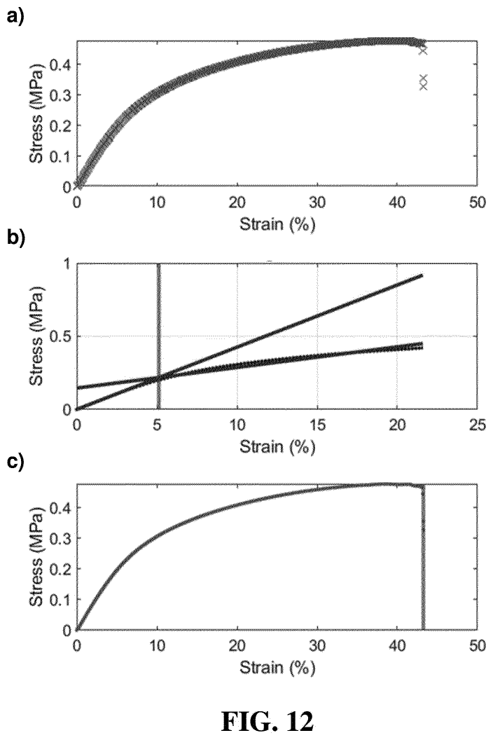

Young's modulus is calculated as described in the example D.3. (FIG. 12) of the present application with the parameters mentioned above using the following equation:

$${E}_{M e m} = \frac{σ}{ε}$$

E_{Mem} is the Young's modulus of the measured membrane scaffold, σ is the measured tensile stress, and ε is the axial strain in the linear elastic region of the membrane scaffold. Tensile strength is the maximum stress that the membrane scaffold can withstand while being stretched or pulled before breaking and can be measured as described in the example D.3. of the present application (FIG. 12).

According to one particular embodiment, the polymer support does not have a fibrous structure.

The polymers according to the present disclosure that can be used in the scaffold are polyester derived poly (α-hydroxy acids).

Non-limiting examples of polyester derived poly (α-hydroxy acids), in particular biosourced and/or biodegradable polyesters, that can be used to form the scaffold are homopolymers and copolymers of hydroxy acids, such as polylactic acid (PLA), polyglycolic acid (PGA), lactic and glycolic acid copolymers (PLGA), poly(3-hydroxybutyrate), poly(4-hydroxybutyrate), poly(3-hydroxyvalerate), poly(5-hydroxyvalerate), poly(3-hydroxypropionate), poly(3-hydroxyhexanoate), poly(3-hydroxyoctanoate), poly(3-hydroxyoctodecanoate); polycaprolactone; homopolymers and copolymers of poly(butylene succinate) and of poly(butylene adipate) such as Poly(butylene succinate-co-butylene adipate) (PBSA), Poly(butylene succinate) (PBS); and polyhydroxyalkanoates (PHA) and their derivatives polyhydroxyesters of 3-, 4-, 5-, and 6 hydroxyalkanoic acid and mixtures thereof.

In a preferred embodiment, said scaffold comprises between 1 to 50% of the polyester polymer (w/w), preferably between 5 and 20% (w/w).

Polyesters have interesting properties from a mechanical point of view, they are compatible with cell culture. They can be biosourced since they can be produced from agricultural waste and/or can be fully biodegradable. However, polyesters are hydrophobic and impermeable to oxygen and do not allow the diffusion of molecules after reticulation.

Thus, to promote gas exchange, said oxygen impermeable polymer scaffold can further comprise nanopores and micropores which have a diameter of less than 20 μm, in particular from 0.1 μm to 10 μm, more particularly from 2 to 8 μm.

Said scaffold composed of oxygen impermeable polymer such as polyester polymer and comprising micropores can be an asymmetric scaffold or symmetric scaffold.

The honeycomb macroporous polymeric scaffold also named herein micro/macroporous membrane scaffold according to the present disclosure can be accomplished by inversion phase (e.g., Non-solvent Induced Phase Separation (NIPS)), where the exchange between the solvent and the non-solvent control the pore formation. The escape of the solvent and the entry of the non-solvent between the reticulating chain polymer in the presence of a macroporogen creates micro and macropores into the polymer to allow the creation of a dual porosity in the scaffold according to the present disclosure.

Phase inversion are method well-known in the art and included for examples Non-solvent Induced Phase Separation Process (NIPS), Thermally Induced Separation Process (TIPS), Vapor Induced Phase Separation process (VIPS), and Polymerization Induced Phase Separation (PIPS).

TIPS is a process wherein a polymer solution is formed at high temperature with a high-boiling point solvent and cooled to induce phase separation and polymer solidification. The microporous membranes are obtained after the extraction of the diluent.

In the VIPS process, a cast film consisting of polymer and solvent is exposed to a vapor atmosphere of nonsolvent molecules, typically water. The precipitation of the polymer occurs due to the penetration of the vapor into the film, which eventually forms a symmetric porous membrane without a dense skin layer. The thermodynamic properties of the casting solution in the NIPS and VIPS methods are almost similar, suggesting that the VIPS method should produce membranes with morphologies similar to those produced using the NIPS method.

PIPS process is a phase separation which occurs in a multicomponent mixture induced by the polymerization of one or more components. The increase in molecular weight of the reactive component renders one or more components to be mutually immiscible in one another, resulting in spontaneous phase segregation. The morphology of the final phase separated structures are generally random owing to the stochastic nature of the onset and process of phase separation.

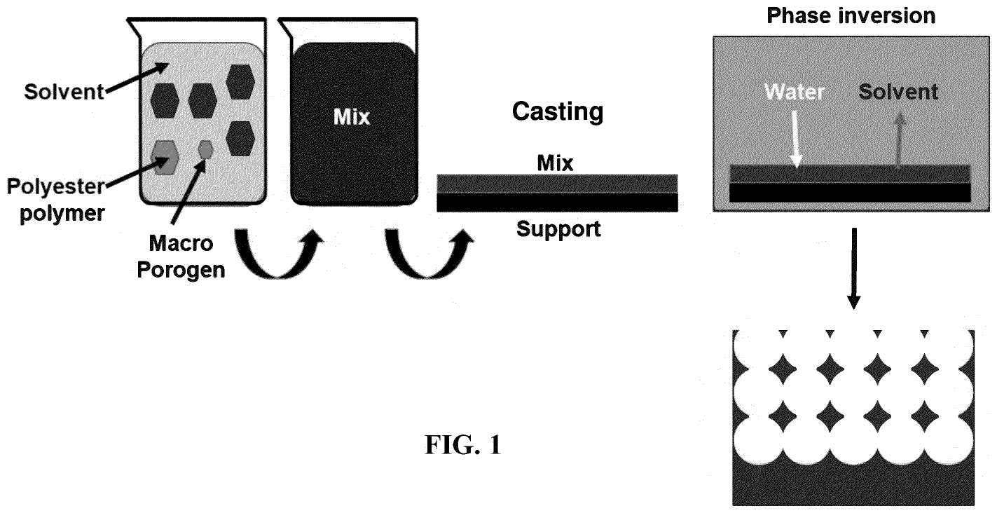

In particular, the scaffold can be produced by the steps of: preparing a solution of said polymer comprising said polymer and at least one solvent for the polymer as described above; adding a solid porogen to the ready solution of the polymer under homogeneous stirring; pouring said solution containing the polymer and the porogen onto the solid support; and introducing said solution of the polymer into non-solvent solution (e.g. ethanol and water), and finally immersing the porogen/polymer composite in a porogenic solvent to dissolve porogen (FIG. 1).

According to the methods described above, the solid substrate on which said solution of the polymer is deposited can in particular be made of glass, metal or plastic resistant to solvents, such as polytetrafluoroethylene (Teflon®), nylon 6,6 or poly(ethylene terephthalate).

Examples of suitable porogen include inorganic salt crystals such as sodium chloride crystals or potassium chloride and amorphous materials such as poly(ethylene glycol) (PEG), polyvinylpyrrolidone, crystals of sucrose, gelatin spheres and paraffin spheres.

In particular, the concentration of porogen in the solution of polymer may in particular be from 10% to 98%, more particularly from 30% to 95% by weight relative to the weight of the polymer.

The solvent of the polymer can be chosen easily by one skilled in the art, and can be selected as non-limiting examples among: N, N-dimethylformamide, N-methylpyrrolidone, ethyl lactate, ethyl acetate, dimethylacetamide, dimethyl sulfoxide, chloroform and mixtures thereof, and non-solvent may in particular be water.

Since the kinetics of coagulation is different at the face in contact with the non-solvent and at the face in contact with the substrate, the formation of pores of different sizes is observed. The direct contact of the polymeric solution with non-solvent will lead directly to a smooth nanoporous face while the slow diffusion of the non-solvent in between the substrate and the collodion will lead to a slow departure of solvent creating a polymer-rich phase and polymer-poor phase. After coagulation, the polymer-rich phase forms the solid mass of the scaffold and the polymer-poor phase forms an interconnected network of pores of different sizes opened at the surface and progressing in the deep of the scaffold. The honeycomb macroporous polymeric scaffold obtained by the NIPS process is an asymmetric scaffold which has a network of interconnected macropores and micropores. Said honeycomb macroporous scaffold presents a smooth face and a porous face, wherein said porous face comprises macropores opened at the surface of diameter between 100 and 280 μm, in particular between 110 and 225 μm with a surface pore area distribution comprised between 40% to 95%, preferably between 50% and 95%, 55% and 95%, 60% and 95%, 70% and 95%, more preferably 75 and 95% and pore wall thickness below 70 μm, preferably below 60 μm, more preferably comprised between 2 and 50 μm, wherein said pores extend deeply from the porous face to the smooth face.

By asymmetric honeycomb macroporous polymeric scaffold, it is intended a scaffold wherein macropores of diameter between 100 and 280 μm, in particular between 110 and 225 μm are not present throughout the whole thickness of the scaffold. In particular, said asymmetric scaffold presents a first face (porous face) which comprises macropores of diameter between 100 and 280 μm, in particular between 110 and 225 μm with a surface pore area distribution comprised between 40% to 95%, preferably between 50% and 95%, 55% and 95%, 60% and 95%, 70% and 95%, more preferably 75 and 95% and preferably pore wall thickness below 70 μm, preferably below 60 μm, more preferably comprised between 2 and 50 μm, and the second opposite face (smooth face) containing only nanopores of diameter below 10 nm, more precisely 5 nm.

In another particular embodiment, the honeycomb macroporous polymer scaffold is a symmetric scaffold.

By symmetric scaffold, it is intended a scaffold wherein macropores of diameter comprised between 100 to 280 μm, in particular between 110 and 225 μm with a surface pore area distribution comprised between 40% to 95%, preferably between 50% and 95%, 55% and 95%, 60% and 95%, 70% and 95%, more preferably 75 and 95% and preferably pore wall thickness below 70 μm, preferably below 60 μm, more preferably comprised between 2 and 50 μm are homogeneously distributed throughout the thickness of the scaffold, and in particular wherein said scaffold has two macroporous faces, in particular wherein said faces both comprise macropores of diameter comprised between in particular between 100 and 280 μm, in particular 110 to 225 μm with a surface pore area distribution comprised between 40% to 95%, preferably between 50% and 95%, 55% and 95%, 60% and 95%, 70% and 95%, more preferably 75 and 95% and pore wall thickness below 70 μm, preferably below 60 μm, more preferably comprised between 2 and 50 μm.

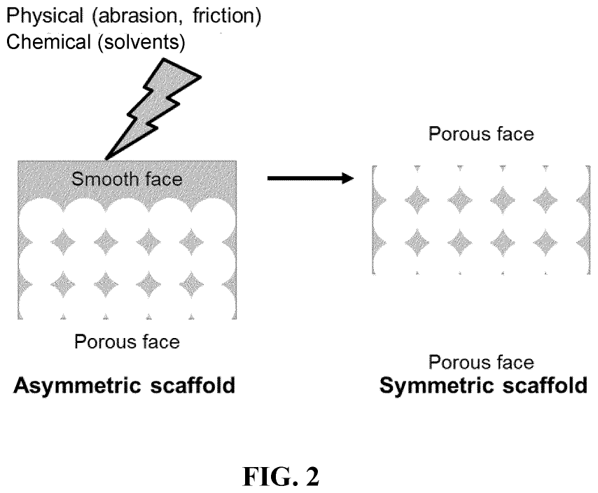

A symmetric honeycomb macroporous scaffold can be accomplished by any methods known by one skilled in the art. Non-limiting examples can be: a) physical abrasion of the smooth face, and/or b) chemical dissolving of the smooth face using polyester polymer solvents (FIG. 2).

In particular, the polyester symmetric membrane scaffold can be produced by the steps of: fabrication of a polymer asymmetric scaffold as described above, physical abrasion and/or chemical dissolving of the smooth face using polyester polymer solvents, and finally immersing the scaffold in water to remove the said physical/chemical agents.

In order to functionalize the surface of the scaffolds designed as described above and increase the cytocompatibility of the scaffold, a bioactive molecule can be grafted at the surface of said scaffold. Said bioactive molecule can be chosen from polysaccharides such as cellulose, pectin, pullulan, keratan, hyaluronan, chondroitin sulfates, chitosan and heparin; proteins such as fibrinogen and collagen, in particular soluble collagen; peptides such as those known for their cell adhesion capacity, for instance those containing the arginine-glycine-aspartic acid (RGD) or arginine-glycine-aspartic acid-serine (RGDS) sequence; and mixtures thereof. The bioactive molecules can be grafted by any methods well-known in the art.

According to one particular embodiment, the bioactive molecules are collagen. Indeed, collagen allows cell adhesion as collagen specifically interacts with integrin receptors expressed at the surface of skin-derived cells. In order to graft collagen at the surface of the scaffold, in a particular embodiment, said scaffold can be treated with plasma generated in air or oxygen, to add electronegative silanol groups. Collagen is then grafted at the surface of the scaffold by immersing said scaffold in a solution comprising collagen, followed by rinsing, preferably with phosphate buffered-saline solution (PBS).

According to one preferred embodiment, the bioactive molecules can be glucides and/or glucide derived molecules such as glycosaminoglycans (e.g., heparin sulfate, heparin, chondroitin sulfate, dermatan sulfate, keratan sulfate, hyaluronic acid). By virtue of its unique biophysicochemical properties (viscoelasticity, high water retention capacity, capacity to interact specifically or nonspecifically with various proteins), glycosaminoglycans play a fundamental role in extracellular matrix organization and homeostasis.

In order to graft glucides and/or glucide-derived molecules (e.g., glycosaminoglycan) at the surface of said scaffold, the scaffold can be firstly pretreated to bring positive charges, preferably positive amine group to the surface of said scaffold before being functionalized with negatively charged molecules. Said positive amine group may be added by treating said scaffold with plasma generated in oxygen or air and immersing said scaffold in polylysine solution, by treating said scaffold with plasma generated in azote or by aminolysis reaction. Once the surface of the scaffold has been treated to be positively charged, said scaffold can be functionalized with glucides and/or glucide-derived molecules by immersing the scaffold in a solution comprising the said glucides and/or glucide-derived molecules, followed by rinsing, for example with ultrapure water.

According to a preferred embodiment, said positive amine group is added by aminolysis reaction of the polymer using a solution of at least one aliphatic α,ω-diamine. The aminolysis reaction can in particular be carried out by immersing the polymer scaffold in a solution comprising aliphatic α,ω-diamine, preferably by immersing said scaffold with 1,6-hexanediamine solution in propanol or water and mixtures thereof, followed by rinsing for example with ultrapure water.

According to the method of the present disclosure, fibroblasts are seeded onto the polymer scaffold as described above and cultured under suitable growth conditions, in particular in a medium which allows the proliferation and the induction of the secretion of extracellular matrix such as different types of collagen and elastin fibers.

Fibroblasts according to the present disclosure is a cell that synthesizes extracellular matrix such as collagen, elastin, glycoproteins and non-proteoglycan polysaccharides among other extracellular matrix components. Fibroblasts can be mammalian or non-mammalian fibroblasts. The cells can be a single cell type or a combination of cell types.

As the only components that resist to the storage and tanning processes are the collagen and elastin fibers that are secreted by fibroblasts, in a preferred embodiment, fibroblasts are the only cells cultured on the scaffold according to the method of the present disclosure.

Numerous culture mediums are available commercially and are well-known to the person skilled in the art. This medium may be a minimum medium particularly comprising mineral salts, amino acids, vitamins and a carbon source essential to cells and a buffer system for regulating pH. The basal medium able to be used in the method according to the invention includes, for example, but are not limited to, MEM medium, DMEM/F12 medium, DMEM medium RPMI medium, Ham's F12 medium, IMDM medium and KnockOut™ DMEM medium (Life Technologies). Depending on the medium used, it may be necessary or desirable to add glutamine, ascorbic acid, growth factor, one or more antibiotics such as streptomycin, penicillin and/or anti-mycotic.

In another aspect, the present disclosure also relates to a tissue (also named in-vitro skin) obtainable by the cell culture step a) as described above, preferably the tissue comprises fibroblastic cells cultured on a honeycomb macroporous polymeric scaffold as described above comprising macropores of diameter comprised between 100 and 280 μm, preferably between 110 and 225 μm with a surface pore area distribution comprised between 40% to 95% and pore wall thickness below 70 μm.

In a particular embodiment, said scaffold comprises polyester, preferably biodegradable and/or partially or fully biosourced polyester, preferably at a concentration comprised between 1 and 50% (w/w), preferably between 5 and 20% (w/w).

In a preferred embodiment, said tissue comprises fibroblastic cells cultured on a honeycomb macroporous polymeric with macropores interconnected with micropores, preferably said micropores having a diameter of less than 20 μm, preferably less than 10 μm.

In a preferred embodiment, said tissue comprises fibroblastic cells cultured on a honeycomb macroporous polymeric with a tensile strength comprised between 0.1 and 10 MPa, 0.1 and 8 Mpa, 0.1 and 5 Mpa, 0.1 and 2 Mpa, 0.1 and 1 Mpa, preferably between 0.1 and 0.8 MPa, more preferably 0.2 and 0.7 MPa.

In another preferred embodiment, said tissue comprises fibroblastic cells cultured on a honeycomb macroporous polymeric with a Young's modulus comprised between 5 and 1 MPa, preferably 5 and 800 kPa, 5 and 700 kPa, 5 and 600 kPa, 5 and 500 kPa, 5 and 400 kPa, 5 and 300 kPa, 5 and 200 kPa, preferably 5 and 100 kPa, 10 and 80 kPa, 40 and 70 kPa, 20 and 60 kPa, more preferably 30 and 50 kPa.

In another embodiment, said tissue comprises fibroblastic cells cultured on an asymmetric scaffold or a symmetric scaffold as described above.

In another preferred embodiment, said tissue comprises fibroblastic cells cultured on said scaffold obtained by non-solvent induced phase separation processes (NIPS).

In a preferred embodiment, said tissue is obtained after at least one week, preferably two, three or four weeks of culture.

Fibroblast cultures on the scaffold as described above allow to obtain a tissue comprising a dense organized mesh of cells and extracellular matrix, preferably with cell/extracellular matrix grooves from 200 to 400 μm as observed in photonic and/or scanning electron microscopy.

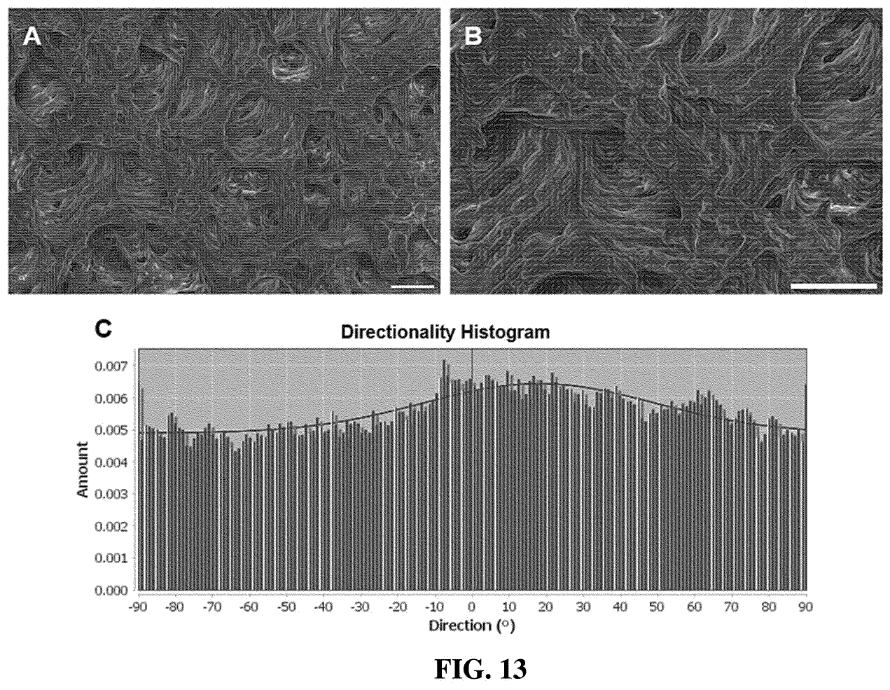

The tissue (in-vitro skin) according to the present disclosure has at least one of the following properties:
the tissue comprises at least 50 μg/mg, preferably at least 60, 70, 80, 90 or 100 μg/mg, more preferably between 50 to 300 μg/mg, again more preferably between 60 to 250 μg/mg of collagen per mg dry tissue. The quantity of collagen is preferably measured by hydroxyproline assay as defined in Example E.1.1 of the present application,
the tissue comprises a percentage of elastin mass in the total dry mass of said tissue of at least 0.3% (w/w), preferably at least 0.4% (w/w). The elastin mass is preferably measured by Elastin assay as defined in Example E.2.1 (Elastin content), in particular by determination of elastin concentration in supernatant of dry tissue digested with oxalic acid,
the tissue comprises a percentage of total fatty acid mass in the tissue total dry mass of less than 1% (w/w), preferably less than 0.1 or 0.01% (w/w), preferably measured by gas chromatography mass spectrometry as defined in example E.6.1 of the present application,
the tissue comprises less than 400 ng/mg, preferably 300 ng/mg, more preferably 200 ng/mg of total glycosaminoglycans (GAG) per tissue total wet mass corresponding to the sum of sulfated GAG and hyaluronic acid (HA), preferably the tissue comprises less than 200 ng/mg of HA per total tissue wet mass, preferably less than 150, 100 ng/mg of HA per tissue total wet mass and/or less than 300 ng/mg tissue of sulfated GAG per total tissue wet mass, preferably 200 ng ECS/mg of sulfated GAG per total tissue wet mass. In a preferred embodiment sulfated GAG is measured by staining with dimethylene blue as described in Example E. 7.1 and/or Hyaluronic acid is measured by addition of acetate-saturated ethanol and cetylpyridinium as described in Example E. 7.1,
the tissue is an isotropic material on the surface, preferably analyzed by 2D Fast Fourier Transform (FFT) observed by SEM as described in Example E.3 Directional analysis of biomaterial fibers in in vitro skin. In particular, said tissue shows the absence of representative peaks with variations of the few percent consistent as illustrated in FIG. 13C,
the tissue comprises less than 0.3, preferably less than 0.2 ppm of extractable and total heavy metal, preferably measured as in Example E.4.1 Heavy metals content
the tissue comprises less than 2 mg/g, preferably less than 1 mg/g of free amine per mass of dry tissue, preferably measured as in Example E.5.1 Quantification of free amino groups,
the tissue has at least two denaturation temperatures (Td) determined by differential scanning calorimetry (DSC) assay, preferably three Td. Said denaturation temperature is preferably measured by wet differential scanning calorimetry (DSC) assay as defined in Example F1.1, more preferably at the constant speed of 5° C./min ranging from 5 to 100° C.

Tissue obtained by the cell culture step as described above is thereafter tanned by any methods well-known in the art to create chemical bonds between the fibers of elastin and collagen to transform it into leather.

A variety of tanning processes may be used to tan leather, including chrome with chrome (III) sulfate tanning agent, tanning using aluminum salts, aldehydes, organic compounds, vegetable tanning using tannins, and tanning with phenolic type and acrylic type polymers of low molecular weight (hereafter “polymer tanning”).

In another aspect, the present disclosure relates to a tanned leather obtainable by the method as described above. As used herein, the term “leather” or “synthetic leather” refers to material obtained from the tanning or chemical treatment of animal skins or tissue comprising collagen, elastin and other components of the extracellular matrix. According to the present disclosure, said tissue is obtained by an in vitro culture step of fibroblasts and comprises a dense mesh of cells and extracellular matrix components.

The tanned leather (in-vitro skin leather) obtainable by the method as described above has at least one of the following properties:
the leather has at least two denaturation temperatures (Td) determined by differential scanning calorimetry (DSC) assay, preferably three Td. Said denaturation temperature is preferably measured by wet differential scanning calorimetry (DSC) assay as defined in Example F.1.1, more preferably at the constant speed of 5° C./min ranging from 5 to 100° C.,
the leather has a total porosity between 30 and 60% preferably measured by mercury intrusion porosimetry as described in Example F.2.1 mercury intrusion porosimetry;
the leather has a water vapor permeability comprised between 0.5 and 5.5 mg/cm²·h, preferably measured at 23° C. in the presence of 50% relative humidity as described in Example F.3.1 Water vapor permeability.

The present disclosure also relates to the use of macroporous scaffold as described above for producing leather, preferably by tanning tissue obtained from fibroblasts culture on said honeycomb macroporous polymeric scaffold, more preferably wherein a surface of said scaffold comprises macropores of diameter comprised between, 100 to 280 μm preferably 110 to 225 μm with a surface pore area distribution on the surface comprised between 40% to 95%, preferably between 50% and 95%, 55% and 95%, 60% and 95%, 70% and 95%, more preferably 75 and 95%. In a preferred embodiment, said surface of scaffold has a pore wall thickness below 70 μm, preferably below 60 μm, more preferably comprised between 2 and 50 μm. In a particular embodiment, said scaffold is a polyester scaffold, preferably honeycomb polyester macroporous scaffold. In another particular embodiment, said scaffold is an asymmetric or symmetric scaffold.

Embodiments of the present invention are described in the following specific examples which are exemplary only and not to be construed as limiting.

### EXAMPLES

### A. Obtaining Asymmetric Honeycomb Polyester Macroporous Scaffold Using NIPS

### 1-Fabrication of Macroporous Honeycomb Polyester Scaffolds (Membrane Scaffold)

Asymmetric polyester membranes are prepared according to the NIPS process. Polymer solution (w_{polymer}/vₛₒₗᵥₑₙₜ) is obtained by dissolving the selected polyester with or without the selected porogen in different mass ratios into the selected solvent at 70° C. for 2 h to ensure their complete dissolution. After bubble elimination, the solution was casted onto a glass plate and spread to form films. The thickness of the casted film was limited by 7 paper wedges on the two lengths of the glass plate. The glass plate carrying the polymer film was then immediately immersed in a Milli-Q water bath at room temperature (i.e., 20-22° C.). After coagulation, the formed membrane was thoroughly washed with Milli-Q water and stored in a desiccator under vacuum at room temperature (FIG. 1).

### 2-Morphological Characteristics of the Polyester Membrane Scaffolds

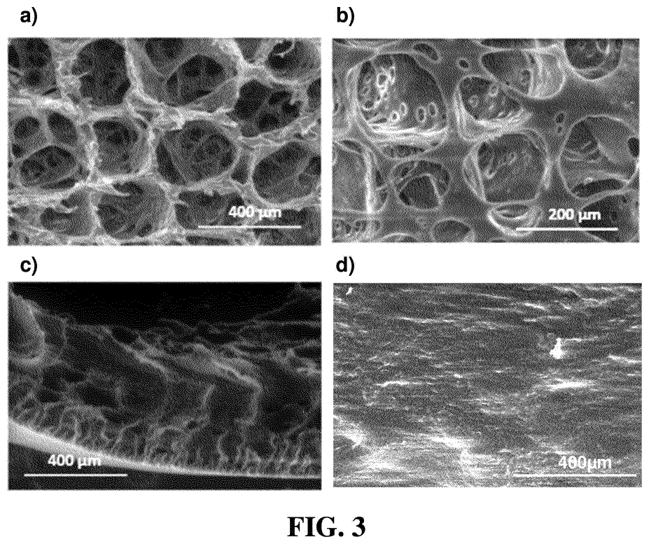

Polyester membranes were prepared by the NIPS process using polymer solutions with various polymer content. For most of the membranes, the two faces presented different aspects to the naked eye indicating the existence of an asymmetric structure. Observations by optical microscopy and by SEM make it possible to examine in more detail the morphology of the two faces (FIG. 3).

These observations confirm the asymmetric structure of the membranes with the existence of a smooth top face (FIG. 3d) and a macroporous bottom face (FIGS. 3a, b) with pores that extend deeply from the porous face to a few nanometers of the smooth face (FIG. 3c).

As expected, using the NIPS process, the coagulation of the polyester polymers occurred when the film of the polymeric solution was immersed in the bath of water which is a non-solvent of the selected polyester polymer. During this process, the polymer solvent, miscible with the non-solvent, was progressively removed, thereby causing gelation of polyester polymer and then its vitrification and its crystallization. More precisely, the top face of the film was in direct contact with water while the bottom face was in contact with the substrate. Therefore, the coagulation phenomenon occurred faster on the top side than on the bottom one. On the top side, the rapid departure of the solvent led to the formation of a polymer concentrated gel which subsequently generated the smooth face after solidification of the membrane. The progressive diffusion of the non-solvent through the polymer gel led to a phase separation into a continuous polymer-rich phase and a dispersed polymer-poor phase that was increasingly marked from the top side to the bottom side of the polymeric film. The evolution over time finally led to the solidification of the polyester-rich phase and to the creation of pores filled with non-solvent by the coalescence of the droplets of the polymer-poor phase. The NIPS process thus makes it possible to manufacture asymmetric membranes comprising macropores in the form of gloves fingers open on one face and extended in the depth of the membrane to form a smooth face without macro/micro pores on the other side (FIG. 3c) (see al tawil et al., European polymer journal. 105, 370-88, 2018).

### 3. Effect of Macro Porogen Addition

The inventors were interested in asymmetric membranes with macropores open to the surface having a sufficiently large diameter (˜140 μm) and a morphology suitable for accommodating fibroblasts and their ECM. Thus, the inventors have added a porogen to better control the pore diameter and the interconnection between the macropores. The membranes showed macropores open on the surface with an increase of the number and the diameter of the macropores and the micropores when the selected macro porogen was added.

Interestingly, membrane observation by SEM (FIG. 3) revealed that in addition to the classical increase in porosity which can be attributed to the extraction of the porogen during the film coagulation step, addition of the macro porogen led to the formation of a dual pore architecture with a network of interconnected macropores and micropores. In these conditions, a well-organized honeycomb macroporous structure was observed (FIG. 3a) and few micropores in the wall of the macropores (FIG. 3b).

### 4. Surface Modification of Polyester Membranes

The polyester membrane is functionalized with glucide derived molecules after an aminolysis reaction. The Coloration with red ponceau after aminolysis and with brilliant green after glucide derived molecules immobilization reveals a successful surface modification of the polyester membrane surface. All colorations were homogeneous on all the surfaces of the samples. Moreover, the untreated scaffolds do not retain the colorations meaning that surface modification is a crucial step to immobilize the HA and to host the cells.

### B. Cell Behavior and Tissue Production in an Asymmetric Honeycomb Polyester Macroporous Scaffold.

### 1. Cell Behavior on Polyester Sponges and Membranes

Cell behavior is stimulated by biochemical (soluble factors), physical cell-cell or cell-ECM interactions and substrate (scaffold) mechanical stimuli. As a result, cells can adhere, proliferate, differentiate, go dormancy, or transform. Here, the inventors highlight the influence of scaffold stiffness on cell behavior in combination with morpho-structural parameters.

PDMS and Polyester sponges obtained by Solvent Casting and Particulate Leaching (SCPL) using either a calibrated sodium chloride (Ref. S5886-5 KG, Sigma) or an organic macropore, and polyester membranes obtained by NIPS using a selected macropore were seeded with fibroblastic cells. These scaffolds were functionalized with glucide derived molecules, seeded with fibroblasts and cultures were maintained for up to 5 weeks.

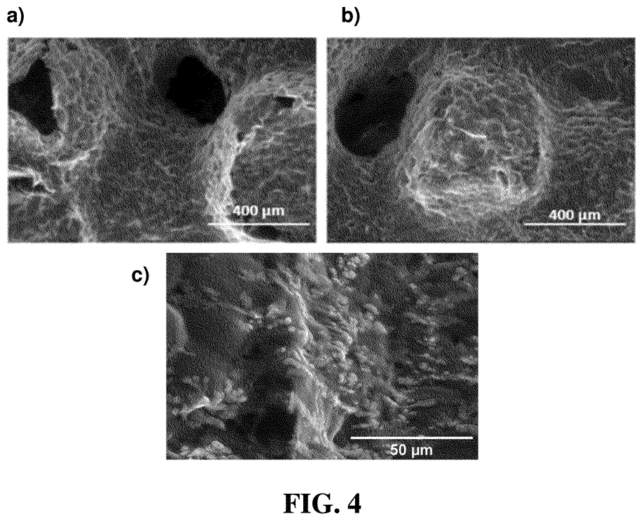

From the first hour of seeding, cells were adherent on both PDMS and polyester sponge surfaces and started to proliferate. SEM images of the polyester sponge (FIG. 4) showed a cell layer lined the entire surface of the sponge: cell bodies (rounded shape cells) were detectable on the entire surface of the sponge and this at different culture times (3 and 5 weeks). The observed cell density was astonishing compared to expected results. As cells were biologically active (viability and proliferation), ECM deposition was low (Dekker et al., Tissue Engineering of Cartilage and Bone: Novartis Foundation Symposium 24. Volume 249. 2003). This result pushed us to hypothesize a probable cross-talk between the morphological and mechanical properties of the scaffold and the observed cell behavior.

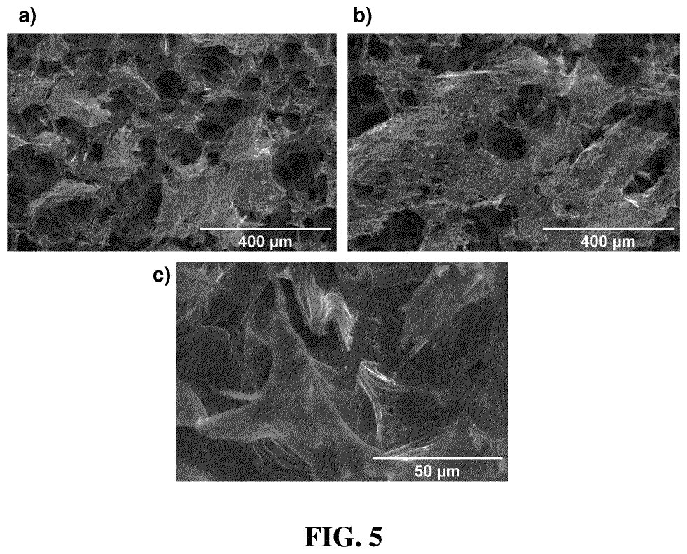

Fibroblasts exhibited a different behavior on the polyester membranes obtained using the NIPS process. Identical to sponge condition, cells were adherent from the first hour after seeding, they proliferated and colonized the entire membrane surface. Very interestingly, SEM images (FIG. 5) showed less cell bodies compared to polyester sponges with a remarkable amount of extracellular fibers spread in the macropores and micropores.

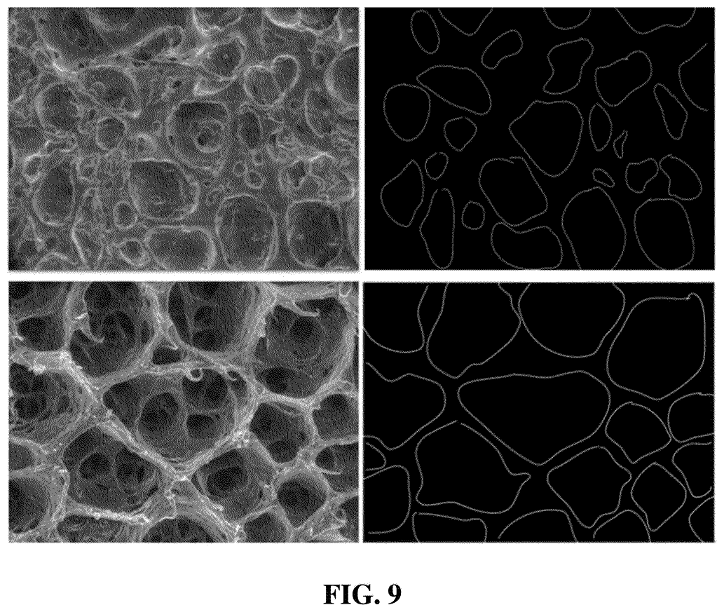

From a morphology point of view, PDMS and polyester sponge scaffolds exhibited a heterogeneous architecture with lower surface porosity and higher wall thickness between macro pores. For the polyester membranes, the homogeneous but irregular distribution of the honeycomb pores gives rise to a well-organized structure with higher surface porosity and lower wall thickness between macro pores (FIG. 9). The presence of the material mass (polyester) in an irregular thick way between the voids in the sponge modifies the mechanical properties, while the thin pore walls in the membrane give rise to a less stiff material with a different degree of elasticity.

Since scaffold stiffness influences the cytoskeletal organization, it also affects cell morphology and therefore the behavior. Various studies indicated that stiffer substrates generally induce a more rounded cell shape closely distributed, whereas soft substrates promote cell spreading (Humphrey J D, Dufresne E R, Schwartz M A. Nat Rev Mol Cell Biol. 2014 December; 15(12):802-12). It is not surprising that these changes in cell morphology are accompanied by changes in cell behavior, including viability, proliferation and/or ECM deposition. The inventors suggest that the presence of thick walls between the sponge pores make it more stiff as material inducing the agglomeration of cells and their colonization of the scaffold in a very narrow way, close to each other.

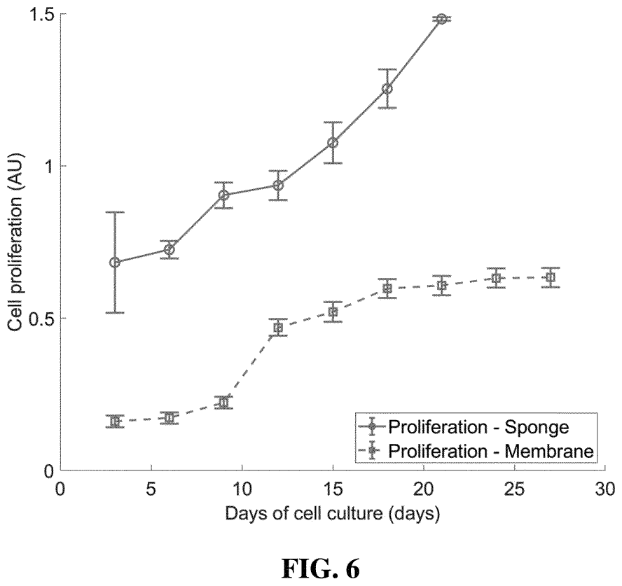

To quantitatively assess the cell proliferation in either polyester sponge or membrane, the inventors tracked cell proliferation using a Cell Proliferation Reagent WST-1 assay used for the nonradioactive, spectrophotometric quantification of cell proliferation and viability of cell populations (Ref. 5015944001, Sigma). As shown in FIG. 6, fibroblasts on polyester membranes (FIG. 6, squared pointed line) have a proliferation curve with a gentle slope indicating an initial phase of cell adaptation (<10 days), followed by a phase of cell proliferation (days 10-15) and a final phase of stationary cell growth (>15 days) that inventors speculate is correlated to the increase in ECM secretion observed in FIG. 5. On the other hand, the proliferation curve of fibroblasts on the sponges (FIG. 6, round and solid line) has an ascendent trend indicating that cells continue to proliferate even after 4 weeks of culture. The maintenance of a proliferation rate after 3 weeks of culture (FIG. 6, round and solid line) reflects higher cell proliferation rate and can be correlated with the low secretion and deposition of ECM observed on the surface of fixed polyester sponges cultured and observed by scanning electron microscopy (FIG. 4). On the other hand, stabilization in the cell proliferation after two weeks of culture of fibroblasts on the polyester honeycomb membrane scaffolds is concordant with the low amount of cell bodies accompanied with a high secretion of ECM observed by scanning electron microscopy (FIG. 5). This antagonism between cell proliferation and ECM secretion is well described by the antagonism of cell proliferation vs. cell specialization (Cooper G M. The Cell: A Molecular Approach. 2nd edition. Sunderland (MA): Sinauer Associates; 2000).

### 2. Tissue Formation in Asymmetric Honeycomb Polyester Macroporous Scaffolds

The micropattern surface, the alignment of ECM fibers, the cells, and the interconnected 3D pore structure are important microstructural features. It has been proven that the micropattern architecture directed multicellular organization and fibrillar collagen deposition, and also affected the alignment and shape of single cells (Gilchrist, C. L., Ruch, D. S., Little, D. & Guilak, F. Biomaterials 35, 10015-10024 (2014)). Additionally, aligned cues were sufficient to direct cell shape, alignment, adhesion, and fibrillar collagen matrix deposition. In this case, the architecture of the polyester sponge is defined as a micropatterned structure (the pores follow the geometric shape of the porogen) while the pores of the polyester membranes have an architecture of ordered honeycomb in surface and aligned gloves in deep. Cells in an ordered alignment are more likely to differentiate, meaning cells are active and secrete a higher amount of ECM (Du, Y., et al. Biomaterials 218, 119334 (2019)).

The relation between architecture (at a macro- and microscale) modulates cell behavior and pushes cells to secrete ECM when proliferating in polyester membranes as shown in FIG. 5. The deposition of the collagen fibers in the pores, the attachment of the collagen fibers to the pore walls will certainly improve the mechanical properties of the construct.

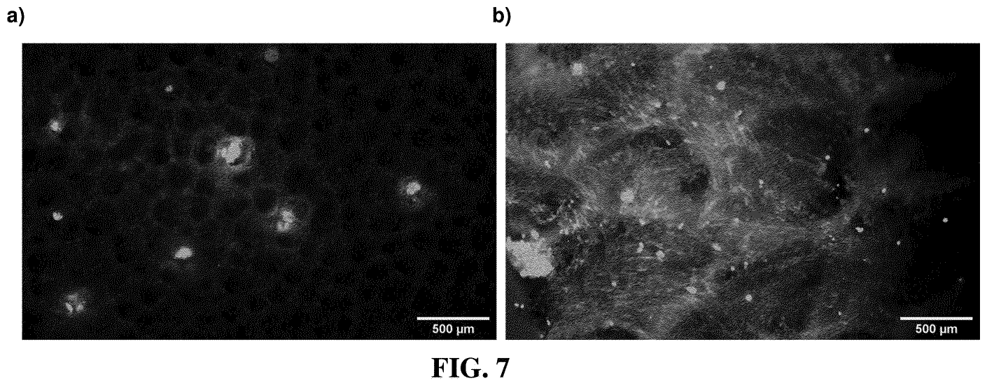

In order to characterize cell proliferation, cell distribution, ECM expression and distribution of ECM fibers in cultured asymmetric honeycomb polyester membrane scaffold constructs, polyester membranes were seeded with fibroblasts and cultured for 1 and 3 weeks, fixed with 3.7% PFA in PBS 1×, and stained using an anti-Collagen 1 specific antibody (Sigma, ref. C2456) combined to a cell-nucleus staining reagent (DAPI) as shown in FIG. 7.

As shown in FIG. 7a, membrane constructs cultured for 1 week show cell bodies proliferating into the membrane macropore structure, with low amounts of collagen-1 secreted and the absence of collagen-1 fibers. This result is consistent with the observation that cells proliferate during the first two weeks of culture in membrane scaffolds as shown on FIG. 6 (round shapes, full line).

In contrast, membrane constructs cultured for 3 weeks show tissue comprising a dense organized mesh of cells and collagen-1 (FIG. 7b), in particular with cell/collagen-1 grooves from 200 to 400 μm reminiscent of rete ridge, protruding from the papillary dermis at the interface of animal dermis and epidermis (Q. Zeng, L. K. Macri, A. Prasad, R. A. F. Clark, D. I. Zeugolis, C. Hanley, Y. Garcia, A. Pandit, D. I. Leavesley, D. Stupar, M. L. Fernandez, C. Fan, Z. Upton, 6.20 Skin Tissue Engineering, Editor(s): Paul Ducheyne, Comprehensive Biomaterials II, Elsevier, 2017, Pages 334-382). This result is consistent with the observation that fibroblast culture on membrane scaffolds do not proliferate and enter in a ECM-secretion cell specialization phase as shown in FIG. 6 (squared shapes, pointed line).

### C. Tanning and Leather Production of Cultured Asymmetric Honeycomb Polyester Macroporous Scaffold.

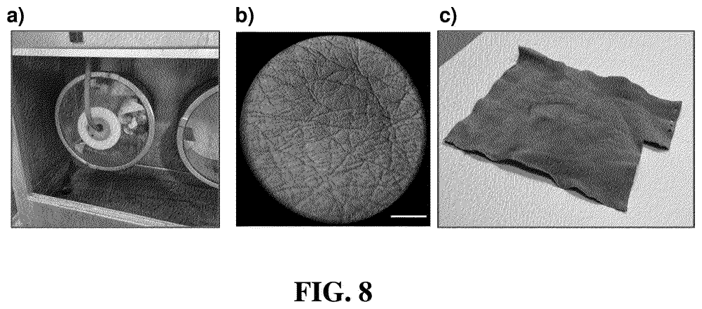

Constructs obtained after culturing fibroblast on the asymmetric honeycomb polyester macroporous scaffold as described above were tanned and retanned using a method known in the art to create chemical bonds between the fibers of elastin and collagen to transform it into leather. Cultured polyester sponges and membranes were tanned and retanned following the said protocol used for traditional animal leather into an experimental tanning drum (FIG. 8a).

Unfortunately, the polyester sponges cultivated with fibroblast for 4-5 weeks became brittle and fragmented during the steps of tanning and retanning. On the contrary, the polyester membrane constructs seeded for either 4 or 5 weeks kept their structural integrity and became leather-like crafted products after tanning and retanning procedure as shown in FIG. 8c. Indeed, asymmetric honeycomb polyester macroporous scaffolds cultured for 4 weeks, tanned and retanned as shown in FIG. 8a showed a leather grain motive characteristic of full grain leather when observed under low-magnification light microscopy (FIG. 8b).

### D. Geometrical and Mechanical Comparison Between Polyester Sponges and Membranes

### 1. Geometrical

From a morphology point of view, polyester sponges exhibited a heterogeneous dispersed porosity (FIG. 9, up) with low surface porosity and higher wall thickness between macro pores. On the other hand, polyester membranes showed a homogeneous but irregular distribution of the honeycomb macro pores gives rise to a well-organized structure with high surface porosity and low wall thickness between macro pores (FIG. 9, down).

### 2. Surface Morphological Analysis of Porous Scaffold: Sponges and Membranes

Scanning Electron Microscope (SEM) images with specific magnification are used for measuring the surface porosity, the pore size and the surface pore area distribution of the membrane. The porous face of the scaffold was imaged by SEM, the corrected magnification was used to standardize the segmentation of the images using ImageJ software (Rasband, W. S., ImageJ, U. S. National Institutes of Health, Bethesda, Maryland, USA, https://imagej.nih.gov/ij/, 1997-2018). The original images (gray/black images) from the outer surface of a porous scaffold are binarized between zero and 255 pixels in which, pixels with low and high luminance were assumed as pore areas and boundaries containing the material, respectively. A proper function was used to eliminate the high and low frequency noise coming from the binarization of pores from background plans (FIG. 9).

In the binarized image each macro pore on the surface area of the hollow sponge or membrane was digitized for auto measurement of the surface pore area, circularity and Feret's pore diameter using the “Analyze Particles” function in ImageJ. From these measurements, it was obtained the surface porosity, the surface pore area distribution and Feret's pore diameter for a statistically representative number of sponges and membrane scaffolds.

The surface porosity of fabricated membranes was calculated by the following equation:

$$Po {r}_{s u f} = {\int }_{0}^{h} \frac{( {A}_{P} ) z * d z}{{A}_{t}}$$

where: At is the total area of image, Ap is the area porosity at distance z, h is height of image.

Surface pore area distribution (pore's numbers/area) is defined by the distribution of the number of labelized pores on the chosen surface. Feret's pore diameter is the longest distance between any two points along the pore boundary, also known as maximum caliper or maximum diameter.

An additional analysis of the architecture of the sponge and membrane scaffold was performed using the ND plugin in ImageJ as described by Haeri, Morteza & Haeri, Mohammad. (2015). Journal of Open Research Software. 3. 10.5334/jors.bn. ND was used to calculate the distance between pores and determine the pore-to-pore wall thickness. The distance between the pores, ie. average surface area between the pores was obtained from the calculation of the distance between the centers of 3 neighboring pores diminished by the sum of the radius of each pore. The centroid of a pore is determined digitally by determining the center of the geometric pore boundary corresponding to a pore. The calculation of the wall distance between the pores was obtained from the distance of the centroid of the pores corrected by the area of pores (Haeri, Morteza & Haeri, Mohammad. (2015). Journal of Open Research Software. 3. 10.5334/jors.bn).

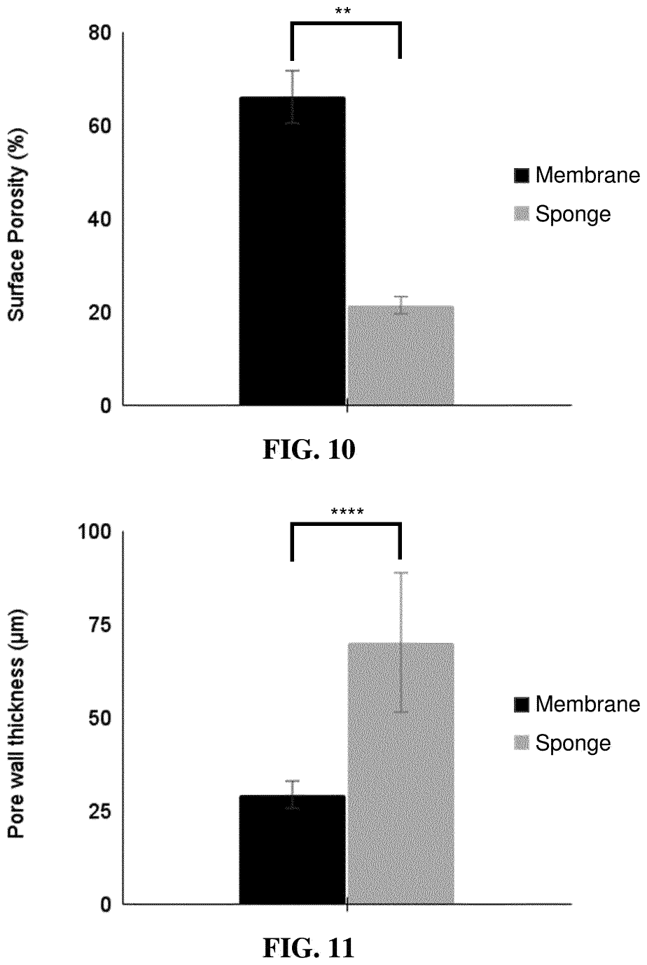

As shown in FIG. 10, surface porosity of membrane scaffolds is 3 times higher than those observed for sponge scaffolds with a median±MAD surface porosity of membrane scaffolds of 66.11±8.53 compared to 21.49±8.65 in sponge scaffolds. Moreover, as shown in FIG. 11, pore wall thickness of membrane scaffolds is 2.4 times lower than those observed for sponge scaffolds with a median±MAD average wall thickness of membrane scaffolds of 29.37±12.40 compared to 70.15±26.60 in sponge scaffolds.

The presence of the material mass in an irregular thick way between the voids in the sponge in addition to a low interconnected porosity modifies the mechanical properties and the communication between cells respectively. A high interconnected porosity like in membranes allows cells to communicate and align deposition of ECM fibers. Moreover, a thin pore wall in the membrane would give rise to a less stiff material with a different set of mechanical properties. Since scaffold stiffness influences the cytoskeletal organization, it also affects cell morphology and therefore the behavior. Various studies (Yeung T, et al. Cell Motil Cytoskeleton 2005; 60(1): 24-34; Gaudet C, et al. Biophys J 2003; 85(5): 3329-35) indicated that stiffer scaffolds with high porosity generally induce a more rounded cell shape closely distributed, whereas soft substrates promote cell spreading. It is not surprising that these changes in cell morphology are accompanied by changes in cell behavior, including ECM deposition. The presence of rigid walls in between the sponge pores can make it more stiff as material and can induce the agglomeration of cells and their colonization of the scaffold in a very narrow way, close to each other. In this provision, cells would establish junctions allowing their survival while minimizing their secretion activity and have a low metabolism while maintaining a high proliferation rate. In this way, cell behavior on polyester sponges and membranes is closely correlated with the morpho-mechanical parameters (porosity, pores, pore wall thickness) of the said scaffolds. This correlation directly controls the tanned construct properties.

### 3. Mechanical Analysis of Asymmetric Honeycomb Polyester Macroporous Scaffolds

Several polyester membrane scaffold samples were tested for their mechanical properties. Different parameters such as thickness and different polymer/porogen formulations and combinations were tested. Each combination was tested wet.

Each sample was cut with press knives as specified in ISO 2419-2012 to create a standard tensile specimen. Environment conditions are ambient temperature and humidity. The samples are shaped like a dog bone. The tested section is 10 mm wide and 50 mm long. The thickness depends on the sample preparation.

The thickness of each sample was measured following the ISO 2589:2016 standard. Thickness is measured with a digital thickness gauge J-40-L manufactured by Checkline that complies with ISO 2589:2016. Three measurements were made, distributed over the sample and averaged.

Physical and mechanical tests on the scaffolds to measure tensile strength and Young's modulus Tensile tests were performed following the ISO 3376:2020 standard. Instron 34SC-1 system is used to perform the tests. It is equipped with an Instron 50N load cell (CAT. NO.: 2530-50N) and Instron manual tightening grip (CAT. NO.: 2710-203). The speed is imposed during the test and set at 100 mm/min. Strength and displacement measurements are recorded.

Stress applied during the test is calculated by the following equation:

$$Stress = \frac{F}{{S}_{T S}} = \frac{F}{W * {T}_{T S}}$$

where: F is traction force applied by the system over the surface of the tensile specimen; S_{TS} is the minimal surface of the tensile specimen, and W and T_{TS} are the width and the thickness of the tensile specimen.

Elongation is calculated with the initial length and displacement on the traction axis. Representative results of the traction mechanical tests performed on polyester scaffolds following the ISO 3376:2020 standard are shown in FIG. 12. The raw stress/strain curve was obtained and displayed in FIG. 12a. Elastic limit was defined as the lowest stress point at which permanent plastic deformation can be measured and differentiated from elastic deformation as shown in FIG. 12b.

The Young's modulus of the membrane scaffolds was measured using the following equation:

$${E}_{M e m} = \frac{σ}{ε}$$

E_{Mem} is the Young's modulus of the measured membrane scaffold, σ is the measured tensile stress, and ε is the axial strain in the linear elastic region of the membrane scaffold.

Tensile strength is the maximum stress that the membrane scaffold can withstand while being stretched or pulled before breaking as shown in FIG. 12c.

Data obtained from these measurements are: Young modulus, elastic limit and tensile strength. These values will be used to characterize the mechanical characteristics of the asymmetric honeycomb polyester macroporous scaffolds.

Young modulus of membrane scaffolds is measured to be of and average±standard deviation of 40.80±14.21 kPa and the tensile strength of the membrane scaffolds is measured to be of an average±standard deviation of 0.4244±0.1603 MPa.

### E. Characterization of the Tissue (In-Vitro Skin) Obtained by Culture of Fibroblasts on Membrane Scaffold.

### 1. Collagen Content

### 1.1 Hydroxyproline Assay Protocol:

In-vitro skins obtained by 4-week fibroblast culture on membrane macroporous scaffolds and rabbit skins (Shaved rabbit skin (dermis/epidermis)) were cut with an 11 mm diameter cutting tool and dried using an oven (Time: 16 h; T° C.: 50° C.). The samples were weighed in a precision balance. The samples are mechanically pulverized and 100 μL of the hydrolysate was recovered for the determination and dosage of the hydroxyproline present in the sample.

The concentration of hydroxyproline present in the sample is determined following the protocol provided by the manufacturer (ref. MAK-357, Sigma Aldrich). A standard curve is obtained and the equation of the curve and the regression coefficient R2 is obtained by linear regression of the standard curve.

The concentration of hydrolyzed hydroxyproline (μg/μL) is calculated using the following formula:

$$Hydrolyzed Hydroxyproline = ( B / V ) \times D$$

with B: amount of hydroxylyzed hydroxyproline calculated from the standard curve (μg); V: volume of sample hydrolysate added to the well (in μL); D: the dilution factor of the post-hydrolysis sample.

The concentration of hydrolyzed can therefore be converted to its collagen equivalent by multiplying by the factor 7.49 (R. B. Neuman and M. A Logan. J. Biol. Chem. 184, 299, (1950)).

### 1.2 Results

To assess collagen secretion in in-vitro skin, samples were seeded and cultured for four weeks in the presence of collagen activation. Quantification of hydroxyproline was performed weekly during the four weeks of culture according to the previously described protocol. The amount of collagen in in-vitro skin after 4 weeks of culture determined by the hydroxyproline assay is 106.53 μg/mg while the rabbit skin is 69.03 μg/mg.

### 1.3 Conclusion

The analysis of hydroxyproline in biological tissues is the reference method for measuring the content of collagen extracted from different tissues (G. F. Caetanoa, M. Fronzab et al. Pharmaceutical biology, vol. 54, NO. 11, 2555-2559, (2016)). However, this method poses a difficulty related to the extraction of collagen from the analyzed tissue. Thus, measurements of collagen concentration by hydroxyproline assay in dry animal skins vary between 5 and 30% of the total dry matter (G. F. Caetanoa, M. Fronzab et al. Pharmaceutical biology, vol. 54, NO. 11, 2555-2559, (2016)).

Caetano et al. used the hydroxyproline assay to determine collagen in rat skins (G. F. Caetanoa, M. Fronzab et al. Pharmaceutical biology, Vol. 54, No. 11, 2555-2559, (2016)). The hydroxyproline assay was normalized to the mass of the dried skin before extraction and assay. This study shows a ratio of approximately 6 μg/mg hydroxyproline per mg dry skin, which represents 45 μg/mg collagen per mg dry skin or a mass ratio of 4.5% in the skins analyzed.

Parsons et al. also used the hydroxyproline assay to determine collagen in mouse skin (Parsons K K, Maeda N, Yamauchi M, Banes A J, Koller B H. Am J Physiol Endocrinol Metab. 2006 June; 290). The hydroxyproline assay was normalized to the mass of dry skin before extraction and assay. This study shows a ratio of approximately 28 μg/mg hydroxyproline per mg dry skin, which represents 210 μg/mg collagen per mg dry mouse skin, a mass ratio of 21% in the skins analyzed.

Taking these results into account, the inventors produce in-vitro skin whose collagen composition is consistent with the extraction and hydroxyproline assay results found in the literature.

Indeed, the present results showed a collagen level of 106.53 μg of collagen per mg of in-vitro skin (dry), a mass ratio of 10.6% in the analyzed samples, which is within the range of results obtained in animal skins via this quantification process. A summary of the results obtained in the scientific literature and those obtained in the present study are shown in Table 1.

TABLE 1
Determination of collagen extraction yields in animal
tissues and in-vitro skins by hydroxyproline assay.

| | | | Rabbit skin | In-vitro skin |
|---|---|---|---|---|
| | Caetano | Parsons | measured in the | measured in the |
| | et al. | et al. | present study | present study |
| Dry mass | 45 | 210 | 69.03 | 106.53 |
| collagen | | | | |
| content (μg/mg) | | | | |

### 2. Elastin Content

The mechanical properties of the skin are defined by its composition. Elastin, which represents only a few percent of the dry mass, is responsible for the mechanical response to low stress. In particular, it dictates the shrinkage after deformation, which contributes to the sensory impression left by the skin after mechanical stress.

The inventors extracted and measured the total elastin content of in-vitro skins. By comparing these results with studies in mammalian skins, the inventors showed that the elastin content extracted in samples of in-vitro skins is in the range of mammalian skins.

### 2.1 Elastin Assay Protocol

In-vitro skins obtained by 4-weeks fibroblast culture on membrane macroporous scaffold with circular (19 mm diameter) or square (17 mm side) geometry were collected, washed with physiological saline solution (DPBS, Thermo ref. 10010), dried in a drying oven and weighed with a precision balance. Dried material was cut with a scalpel and digested with oxalic acid at high temperature, centrifuged to remove debris. The supernatants were collected for analysis. 100 μL of the supernatant was recovered and the elastin concentration is determined in the supernatant with Fastin elastin assay kit (Biocolor LTD. ref. F2000). A standard curve (elastin provided in the kit) is obtained. The equation of the curve and the regression coefficient R2 were obtained by linear regression on the standard curve.

### 2.2 Results

### Calibration of the Protocol

To validate the efficiency of elastin extraction using the protocol under the present extraction conditions, the inventors performed successive extraction tests on three 17 mm square samples of in-vitro skins and determined the amount of elastin present in each of these extracts (Table 2).

TABLE 2
Average elastin mass measured by successive
extraction on three in-vitro skins.

| | Total elastin mass (μg) | | | | | |
|---|---|---|---|---|---|---|
| # Extraction | 1 | 2 | 3 | 4 | 5 | TOTAL |
| Material No. 1 | 113.72 | 62.53 | 58.00 | 38.06 | 4.71 | 277.02 |
| Material No. 2 | 113.81 | 61.09 | 57.26 | 37.79 | 5.06 | 275.01 |
| Material No. 3 | 111.46 | 64.56 | 57.32 | 34.33 | 3.46 | 271.13 |
| Mean | 113.00 | 62.73 | 57.53 | 36.73 | 4.41 | 274.39 |
| Standard | 1.33 | 1.74 | 0.41 | 2.08 | 0.84 | 2.99 |
| error± | | | | | | |

In view of the standard deviation from the fifth extraction onwards (#Extraction 5, Table 2), the inventors decided that four successive extractions are effective in extracting elastin from in-vitro skin using the protocol described previously.

To evaluate the portion of non-specific signal that could be linked to the unseeded scaffold, the inventors performed the same extraction and elastin determination on an unseeded membrane scaffold. The inventors measured an absorbance corresponding to an equivalent of 1.42 μg of elastin. This result allowed to determine the threshold value corresponding to the non-specific signal or the reading background. This value is remarkably close to the value obtained at the fifth extraction of the previous experiment, which confirms that 4 extractions are efficient to extract the totality of the elastin present in the sample using the extraction method defined previously.

### Quantification of the Elastin

To assess total elastin in two samples of the in-vitro skins, 19 mm diameter samples were dried and then weighed. Masses of 50.1 mg and 51.4 mg dry matter were measured for these two samples. Four successive extractions of elastin were performed, for a total volume of 2 mL containing the elastin extracts. The non-specific signal was subtracted from the measurements from the determinations made previously. The samples of in-vitro skin numbers #1 and #2 contain 232.55 and 209.69 μg of elastin respectively, representing 0.45% and 0.42% of their dry mass (Table 3).

TABLE 3
mean weight of elastin measured for two samples of in-vitro skins.

| | Sample | Sample |
|---|---|---|
| | #1 | #2 |
| Weight (mg) | 51.40 | 50.10 |
| Extracted Elastin (μg) | 46.51 | 41.94 |
| Standard deviation (μg) | 0.89 | 0.81 |
| Elastin concentration | 116.27 | 104.85 |
| in the extract (μg/μl) | | |
| Total elastin mass (μg) | 232.55 | 209.69 |
| Mass percentage of elastin | 0.45% | 0.42% |

Turner et al. measured elastin in pig skin samples from different locations using a method comparable to the one described here. The amounts of elastin were normalized to dry mass. The measurements reported in this study range from 2.45 to 11.30 μg/mg, i.e. 0.24% and 1.1% in mass percentage (Turner N J, Pezzone D, Badylak S F. Tissue Eng Part C Methods. 2015 April; 21(4):373-84).

Considering the measurements carried out within the framework of this work, the inventors produced an in-vitro skin wherein elastin composition agrees with the results of extraction and quantification found in the literature. Indeed, using this method of experimental quantification, the inventors measured a mass rate of elastin of 4.35 μg/mg of dry in-vitro skin, corresponding to a mass ratio of 0.43%, when the literature evokes a measurement located between 0.24% and 1.1%. A summary of the results obtained in the study of Turner et al. and those obtained by the present study are shown in Table 4.

TABLE 4
Determination of extraction yields and quantification of elastin
in animal tissues and in non-tanned in-vitro skin materials.

| | Turner et al. | in-vitro skin |
|---|---|---|
| Dry mass content | 2.45-11.30 | 4.35 |
| (μg/mg) | | |
| Percentage of dry | 0.24-1.1% | 0.43% |
| mass (%) | | |

### 3. Directional Analysis of Biomaterial Fibers in In Vitro Skin

2D Fast Fourier Transform (FFT) analysis for the alignment of the biomatter network observed by SEM in in vitro skin was measured using the Directionality plug-in for ImageJ (ImageJ Directionality PlugIn. https://imagej.net/plugins/directionality), following methods previously described in Hamley et al. (Hamley, Introduction to Soft Matter: Polymers, Colloids, Amphiphiles and Liquid Crystals, John Wiley & Sons Ltd, Chichester, UK 2000). The Directionality plug-in calculates spatial frequencies in an image based on a set of radial directions. The method generated normalized histograms revealing the amount of fibers present between 0° and 180° with a bin size of 1° as detailed in Deravi et al. (Deravi, et al. (2017), Macromolecular Materials and Engineering. 302). Representative images of the surface of in vitro skin samples analyzed showing orientation vectors of the biomaterial fibers and the histogram of the frequency distribution of fiber directionality are shown in FIG. 13.

The result of the directionality histogram analysis of the biomaterial orientation in the surface of the in vitro skin shows the absence of representative peaks with variations of the few percent consistent with an isotropic material (FIG. 13C).

In contrast, animal skins are anisotropic materials with mechanical and structural weaknesses in particular directions relative to the symmetry axes of the animals at the source of the skins. The isotropic surface of in-vitro skin leathers is explained by their production method, which allows a high degree of control over the parameters that influence cell growth, in contrast to the growth of hides in animals. This difference from conventional animal hides simplifies the use of hides, which do not require special considerations regarding the orientation of cuts on their surface.

### 4. Heavy Metals Content

The presence of hazardous substances in excessive quantities in clothing materials can pose a threat to consumer health, particularly if the products are intended for children and/or in direct contact with human skin. As heavy metals are one of these hazardous substances, thresholds for heavy metal content have been set in standards applied in different jurisdictions. These standards are intended to limit consumer exposure to heavy metals and to ensure a product life cycle that is compatible with environmental issues.

### 4.1 Materials and Methods

The quantification of metals was carried out by an external reference research laboratory (Contract Research Organization, CRO) Eurofins BLC (UK) on in-vitro skin (size: 26×43 cm). The determination of heavy metal content in in vitro skins was performed following international standards to measure: a) Total heavy metal content on digested samples, and b) Extractable heavy metal content with an artificial acidic sweat solution. Table 5 shows the limit values for total and extractable heavy metal content of acceptable materials for commercial leathers.

Sampling was carried out in accordance with BS EN ISO 2418-2017 which specifies the method of sampling (location of the piece of leather), labeling and marking of samples to ensure identification. It is applicable to all types of mammalian derived leather, regardless of the tanning used.

Metal screening was performed in accordance with BS EM ISO 17072-2:2019 and CPSC-CH-E1001-08.3 by atomic absorption spectrometry (AAS) using prior microwave digestion of leather. The detection limit for heavy metals tested using these methods is 0.1 ppm or mg/kg. Where necessary, samples were conditioned and tested at 23° C. and 50% relative humidity (standard atmosphere) according to BS EN ISO 2419-2012.

The determination of extractable metals was performed in accordance with BS EN ISO 17072-1:2019 by atomic absorption spectrometry (AAS) using an extraction with an acidic artificial perspiration solution as a preliminary step. This method allows the determination of extractable metals in leather; it is not specific to a compound or to the oxidation state of the metals. This method is particularly suitable for the determination of extractable chromium in chromium tanned leather. Where necessary, samples were conditioned and tested at 23° C. and 50% relative humidity (standard atmosphere) according to BS EN ISO 2419-2012. The detection limit for heavy metals tested using these methods is 0.1 ppm or mg/kg.

Concentrations of chemicals per unit mass in solids are usually measured in units of chemical mass (milligrams, mg or micrograms, μg) per unit of total mass (kilogram, kg), i.e. mg/kg or μg/kg. Sometimes these concentrations are expressed in parts per million (ppm) or parts per billion (ppb): 1 ppm=1 mg/kg and 1 ppb=1 μg/kg. A measurement of 6 mg/kg is equivalent to 6 ppm or 6,000 ppb or 6,000 μg/kg.

TABLE 5
Extractable content limit values (total content limit value) of
heavy metals in parts per million (ppm) for commercial leathers.

| | | Limit value of extractable |
|---|---|---|
| | | content (limit value of |
| | | total content) |
| CAS number | Metal | ppm or mg/kg |
| 7440-47-3 | Chromium (Cr) | 2 |
| 7440-48-4 | Cobalt (Co) | 4 |
| 7440-50-8 | Copper (Cu) | 50 |
| 7439-92-1 | Lead (Pb) | 1 (90) |
| 7439-97-6 | Mercury (Hg) | 0.5 |
| 7440-02-0 | Nickel (Ni) | 1 |
| 7782-49-2 | Selenium (Se) | 500 |
| 7440-36-0 | Antimony (Sb) | 30 |
| 7440-38-2 | Arsenic (As) | 0.2 (100) |
| 7440-39-3 | Barium (Ba) | 1000 |
| 7440-43-9 | Cadmium (Cd) | 0.1 (40) |

### 4.2 Results

The extractable and total heavy metal content were quantified in in vitro skin. The results are presented in Table 6 (extractable content) and 7 (total content), respectively.

TABLE 6
Results of the measurement of extractable heavy metals in in vitro skin.

| | Extractable heavy metal content | | | | | | | | | | | |
|---|---|---|---|---|---|---|---|---|---|---|---|---|
| | (ppm) (BS EN ISO 17072-1: 2019) | | | | | | | | | | | |
| | Detection limit 0.1 ppm | | | | | | | | | | | |
| Req. | <30 | <0.2 | <1000 | <0.1 | <4.0 | <50 | <1.0 | <1.0 | <500 | <2.0 | <0.5 | — |
| ppm | | | | | | | | | | | | |
| Comp. | Sb | As | Ba | Cd | Co | Cu | Pb | Nor | Go to | Cr | Hg | — |
| Uncert. | — | — | — | — | — | — | — | — | — | — | — | — |
| (+/−) | | | | | | | | | | | | |
| (%) | | | | | | | | | | | | |
| S1 | <0.1 | <0.1 | <0.1 | <0.1 | <0.1 | <0.19 | <0.16 | <0.1 | <0.1 | <0.1 | <0.1 | PASS |

TABLE 7
Results of the measurement of total heavy metals in in vitro skin

| | Total heavy metal content - Total digestion (ppm) | | | |
|---|---|---|---|---|
| | BS EN ISO 17072-1: 2019, CPSC-CH-E1001-08.3 | | | |
| | and E1002-08.3 Detection limit 0.1 ppm | | | |
| Req. | <90 | <40 | <100 | — |
| ppm or mg/kg | | | | |
| Uncert. | 2.70% | 3.60% | 4.30% | — |
| (+/−) (%). | | | | |
| Sample | Lead | Cadmium | Arsenic | — |
| S1 | <0.1 | <0.13 | <0.1 | PASS |

### 4.3 Discussion

The physical characteristics of leathers may vary depending on the manufacturing methods and the structure of the chemicals used during manufacturing. The pH values of leather can vary depending on the pH values of the chemicals used in its manufacture, the additives used in the final stages of the leather manufacturing process and the proportions in which they are used.

The total amount of inorganic matter in hides can be determined by the ash method, and should not exceed 2.5%. In addition, 0.5% mineral matter may be found in natural hides depending on the nutritional status of the animal. When examining the amount of minerals in in vitro skins, it was found that the amount of heavy metals was negligible, only copper and lead were present at more than 0.1 ppm or mg/kg. The presence of copper may be related to the use of cell media, but the origin of the lead traces was difficult to identify. It was found that all values in our research were below the limit values for heavy metal content for commercial leathers.

Today, consumer demand is moving towards materials that do not contain harmful substances and have a reduced environmental footprint. There is therefore a need to produce leather products that contain little or no heavy metals, especially in products that come into contact with human skin. This study found that the heavy metal contents in total in vitro skins and extractable skins using artificial sweat solutions were below the limit values for commercial leathers (Table 5).

This makes in vitro skins a viable alternative for use in commercial leather manufacturing due to its low heavy metal content. In conclusion, it is expected that in the future, the trend of demand for environmentally friendly products will have a greater effect on leather producers, and that the proposed limit values will be lowered. Therefore, the leather industry, and in particular the processing of natural leather, has to prepare itself for these new limit values and for future transformations. In this context, alternatives such as those proposed by the present study is a responsible approach without sacrificing the quality of the products manufactured using animal skins.

### 5. Quantification of Free Amino Groups

### 5.1 Materials and Methods

Proteins from in vitro skins were extracted by sonication (Sonicator Q700 Qsonica) (50% power, 14 s). The proteins were stained by adding 0.5 mL of 0.01% (w/v) TNBSA solution (Thermo Scientific. ref. TS-28997) to 0.5 mL of each sample and incubated at 37° C. for 2 h. The reaction was stopped by adding 0.5 mL of 10% SDS (Sodium bicarbonate buffer solution (0.1M, pH 8.5) solution and 0.25 mL of 1N HCl solution.

Absorbance of solutions is measured at 335 nm. The quantitative number of amines contained in the sample was determined by comparison with the calibration curve established with the glycine solution (10 mg/mL, diluted sequentially to produce a standard range).

### 5.2 Results

Glycine is used to construct the calibration line by measuring the absorbance at 335 nm, corresponding to the formation of a chromogenic compound after reaction with TNBSA, for given concentrations (Table 8).

TABLE 8
Absorbance values at 335 nm as a function of glycine
concentration, used as standard (mg/L).

| Concentration in mg/L | 0 | 5 | 10 | 15 | 20 | 25 | 30 |
|---|---|---|---|---|---|---|---|
| Absorbance at 335 nm | 0.2221 | 0.2970 | 0.3785 | 0.4303 | 0.4879 | 0.5453 | 0.5945 |

TABLE 9
Values of free amine contents in in vitro skins and in the unseeded
membrane scaffold obtained by interpolation of the calibration line.

| | | | | | Concentration | Concentration |
|---|---|---|---|---|---|---|
| | Abs 1 | Abs 2 | Abs 3 | Average | mg/L | mg/g |
| in vitro Skin | 0.4642 | 0.5966 | 0.5970 | 0.5526 | 25.61 | 0.75 |
| Scaffold | 0.3335 | 0.4135 | 0.4423 | 0.3964 | 12.91 | 0.37 |

### 5.3 Discussion

The intensive use of dyeing and tanning in consumer goods such as leather, clothing and toys, can under certain conditions, lead to the formation of total/free amino groups. For this reason, the European Parliament recently adopted the 19^{ème} amendment to Council Directive 76/769/EEC on restrictions on certain substances. The limits set by the legislation, which are currently under review, are 6207 mg/L for total amines and 75 mg/L for free amines. There is an industry method, CEN ISO TS 17234 based on DIN 53316, which can detect twenty of the substances listed in the legislation.

The free amine content of the membrane scaffold and in vitro skins is 12.9 and 25.9 mg/L respectively, which is well below the acceptable limit for free amines set by the directive (75 mg/L). These results are very encouraging and in line with the green technology.

In order to compare the free amine content of in vitro skins and mammalian skins, a bibliographic analysis of the scientific literature on this subject was performed. Soomro et al. quantified the free amine content of human skins using a new method by pre-column derivatization (chromatography) using trifluoro-acetylacetone and isobutyl chloroformate as mobile phases (Soomro S A (2014), Journal of chromatography). This method was applied for the analysis of amines in human skin samples after hydrolysis. In human skin, total free amines were quantified using gas chromatography at approximately 0.9 mg/g human skin.

The total free amine content in in vitro skins is 25.6 mg/L (0.75 mg/g in vitro skin). This result is comparable to that of mammalian skins found in the literature. It is therefore possible to use a wide range of tanning agents, commonly used in the traditional leather industry, to tan in vitro hides.

### 6. Determination of Fatty Acid Content

### 6.1 Material and Methods

Fatty acid quantification was performed by an external reference research laboratory (Contract Research Organization, CRO) Eurofins BLC (UK). Samples were received and processed according to ISO 2418:20179 which specifies the location of a laboratory sample in a piece of leather and the method of labeling and marking laboratory samples for future identification. The sample was reconditioned and tested at 23° C.±2° C. and 50% relative humidity as specified in the standard reference atmosphere requirements of ISO 2419:2012 (for leather).

Quantification of in-vitro skin fatty acid was performed by BLC by coupling: a) fat extraction by supercritical phase chromatography (SFC) 4, followed by b) GC-MS analysis following the protocol described in GB/T 9722-200611.

An internal standard was chosen to determine the major components of fatty acids, volatile reactants and other impurities. Hydrogen/nitrogen was used as the carrier gas and diatomaceous earth (0.18 mm˜0.25 mm) as the stationary phase. The flow rate was determined according to the detection limit of the different fatty acids. The length of the column was set at 2 m and the temperature was maintained at 80° C. throughout the experiment. The process is detailed in ISO 12966-4:201512.

Sampling was carried out in accordance with ISO 2418-20179 which specifies the method of sampling (location of the piece of leather), labeling and marking of samples to ensure identification. It is applicable to all types of mammalian derived leather, regardless of the tanning used.

In vitro skins (26×43 cm) or rabbit skin samples were washed with 1×DPBS physiological saline (Fisher, 12037539), sealed in a vacuum machine, and send to the referring research laboratory (CRO).

Gas chromatography-mass spectrometry (GC-MS) is a sensitive method used to identify and quantify the content of fatty acids. Beforehand, it is necessary to modify the molecules of the carbon chain in order to detect them. Thus, fatty acids are often modified by a derivatization method using an acid, hydrochloric acid being the most used, to form fatty acid methyl esters (FAME). In the GC phase, the samples were isolated according to the different FAMEs. These FAMEs then undergo a strong fragmentation by electron impact ionization in the mass spectrometer and many fragments are formed. For quantification, the largest fragments, which show high abundance and the highest specificity for the individual fatty acid type are selected.

### 6.2 Results

Total fatty acid content was quantified in in vitro skin using the methods described above. The results are presented in Table 10.

TABLE 10
Quantification of fatty acid content in in vitro skin samples

| | | Requirement | Results |
|---|---|---|---|
| Sample | Test | (mg/kg) | (mg/kg) |
| S1 | EMAG | For information | <10 |
| | (GB/T 9722-2006) | only | |
| | | (detection limit: 10) | |

In this study, the measurement of fatty acid content in in vitro skins is low and below the detection limit of measurement of <10 mg/kg.

### 6.3 Conclusions

Various published results indicate that the total fatty acid content of the animals' skin is about 4%. After the use of bath feed, the total fatty acid content increases to about 15% (Mihai A. L; Metrology Promoting Harmonization& Standardization in Food & Nutrition;

3 Imeko Foods). The presence of fatty acids in leather makes it soft and supple. It also prevents the leather from wearing and tearing and makes it waterproof. The total fatty acid content was quantified in in vitro skin. It was found to be less than 10 mg/kg corresponding to 0.001%. This is low compared to the 4% fatty acid content of conventional animal skin.

Some product lines require soft and supple leathers, such as gloves and other products in contact with the skin of consumers. The softness and suppleness of a leather can be modulated during the bath feeding process, during which the individual fibers are evenly coated with the bath feed giving them special properties (Alaskan, A; Polymer Bulletin—Volume 79, doi.org/10.1007/s00289-021-03579-Z).

Since the fat content of in vitro skin is low, it can be precisely modulated during the subsequent bath feeding steps to make it compatible with each type of use in a wide range of products. Among other things, the low grease content of in vitro skins eliminates the need for a degreasing step and the risk of grease spillage and condensation stains on the surface of in-vitro skins, a defect commonly found in conventional hides.

### 7. Determination of Sulfated GAGs and Hyaluronic Acid (HA) Content

Glycosaminoglycans (GAGs), including sulfated GAGs and hyaluronic acid is responsible for the compressive strength of the skin. However, the tanning process requires the removal of GAGs. Their water-holding capacity prevents the circulation of tanning agents in the collagen network; therefore, GAGs must be removed to open this mesh

### 7.1. Hyaluronic Acid and Sulfated Glycosaminoglycans Assay

In vitro skins (with collagen secretion inducer: 19 mm diameter disc) or rabbit skin (170.1 mg wet weight) samples were washed with physiological saline solution (DPBS, Thermo ref. 10010), weighed, cut with a scalpel and digested with proteinase K at 55° C. under agitation, before to be grinded with a potter mill and centrifuged to remove debris and collect supernatant for analysis.

The sulfated glycosaminoglycans content was determined by simultaneous staining and precipitation of sulfated GAGs with dimethylmethylene blue, dissociation of GAG-dye complexes and quantification of the released dye, according to the protocol provided by the manufacturer (Biocolor LTD. ref. B1000). A standard curve (Chondroitin sulfate provided in the kit) is obtained and the equation of the curve and the regression coefficient R2 is obtained by linear regression on the standard curve.

The Hyaluronic acid content was determined by the precipitation of total GAGs by addition of acetate-saturated ethanol, resuspension of HA by addition of cetylpyridinium, refinement of HA isolation by repeating previous steps, and resuspension of HA in distilled water. The concentration of hyaluronic acid present in the sample was determined following the protocol provided by the manufacturer. A standard curve (Chondroitin sulfate provided in the kit) is obtained and the equation of the curve and the regression coefficient R2 is obtained by linear regression on the standard curve.

### 7.2 Results

### Determination of Sulfated GAGs Content

To assess the concentration of GAGs in three in vitro skin samples, 19 mm diameter samples were weighed after draining. The samples were then cut, digested with Proteinase K treatment, and ground. The amount of GAGs was measured following the instructions of the Blyscan kit (B1000). A standard curve was established with a chondroitin sulfate standard. The concentrations measured in the skins, in micrograms chondroitin sulfate equivalent (μg CSE) are reported in Table 11.

TABLE 11
Average mass of sulfated GAGs measured
in three samples of in vitro skin.

| | | Sample | Sample | Sample | Rabbit |
|---|---|---|---|---|---|
| | Scaffold | #1 | #2 | #3 | skin |
| Wet weigh (mg) | 80.6 | 173.2 | 125 | 124.1 | 170.1 |
| Content of extracted | 0.11 | 4.52 | 2.58 | 2.51 | 2.47 |
| sulfated GAG (μg CSE) | | | | | |
| Total mass of sulfated | 0.44 | 18.1 | 10.3 | 10.0 | 98.9 |
| GAGs (μg ECS) | | | | | |
| Standard deviation | 0.24 | 0.12 | 0.12 | 0.16 | N/A |
| (μg ECS) | | | | | |
| Concentration of sulfated | 0.5 | 104 | 81.6 | 78.1 | 580 |
| GAGs | | | | | |
| (ng ECS/mg wet mass) | | | | | |

*The sample volume taken for the quantification of GAGs in rabbit skin is 10 μL instead of 100 μL.

In order to evaluate the portion of non-specific signal that may be related to the unseeded scaffold, the same extraction protocol and determination of the concentration of sulfated GAGs were used on three unseeded scaffolds. The absorbance corresponding to an equivalent of 0.44 μg chondroitin sulfate equivalent (CSE) was measured, and allowed to determine the threshold value corresponding to the non-specific signal, which will be subtracted from each measurement.

### Determination of HA Content

To assess the content of hyaluronic acid (HA) in in vitro skin samples, the same samples were analyzed using the Biocolor Purple-Jelley kit (H1000). The standard curve was established with an HA standard, and the concentrations measured in in vitro skins, are reported in Table 12. The nonspecific signal is 0.04 μg of HA corresponding to the signal of the unseeded membrane scaffold.

TABLE 12
Average HA mass measured in three samples of the in vitro skins

| | Uncultivated | Sample | Sample | Sample | Rabbit |
|---|---|---|---|---|---|
| | Scaffold | #1 | #2 | #3 | skin |
| Wet mass (mg) | 80.6 | 173.2 | 125 | 124.1 | 52.1 |
| HA Extract (μg) | 0.00 | 0.23 | 0.19 | 0.19 | 0.65 |
| Standard | 0.00 | 0.02 | 0.03 | 0.01 | N.D. |
| deviation (μg) | | | | | |
| Total mass of HA | 0.04 | 9.03 | 7.60 | 7.52 | 51.69 |
| (μg) | | | | | |
| HA density | 0.53 | 52.2 | 60.8 | 60.6 | 303.9 |
| (ng/mg wet | | | | | |
| mass) | | | | | |

*The sample volume taken for HA quantification in rabbit skin is 5 μL instead of 10 μL.

### Tests to Determine the Amount of GAGs and HA Per Mg of Dry Mass

To report the measurements of GAGs and HA to the dry mass of the samples, the same procedure was undertaken after oven drying (50° C., 6 h) on in vitro skin samples of the same dimensions. However, after subtracting the nonspecific signal, the total mass of sulfated GAGs is 0.04 μg of ECS, while that of AH is 0.01 μg. This corresponds to densities of 1.3 ng ECS/mg sulfated GAGs and 0.3 ng/mg HA, well below the wet mass determinations, whereas higher densities are expected. Thus, the drying procedure appears to interfere with the quantifications of GAGs in in vitro skins.

### 7.3 Discussion

Sulfated GAGs and HA quantification techniques were adapted to assess the amount of GAGs in in vitro skins. The mass density of GAGs in these skins was measured: a mean density of sulfated GAGs of 87.9±2.2 ng ECS/mg (Chondroitin Sulfate Equivalent) and a mean density of HA of 57.86±4.9 ng/mg were obtained. This density is referred to a wet mass because drying interferes with the measurement. This can be attributed to an extraction problem after drying, for example due to a collapse of the membrane scaffold around the GAGs, or even to degradation during drying.

A study by Armstrong and Bell measured HA in rabbit skin 551 ng/mg wet skin (Armstrong S E, Bell D R. Anal Biochem 2002; 308(2):255-64). This result is 1.67 greater than that presented here, but in the same order of magnitude. The difference could be explained by physiological variations such as the decrease in the amount of HA in the skin with age and location (Barbosa I, et al. Glycobiology 2003; 13(9):647-53; Templeton D M. Connect Tissue Res 1988; 17(1):23-32). Varma et al. report that the distribution of GAGs in different animal skins, including those of rats, is essentially one half HA, another half chondroitin sulfate GAGs, and a small percentage of heparin (Varma R S., Karger; 1982; Fabianek, Herp and Pigman. Comparative Biochemistry and Physiology. Volume 14, Issue 1, January 1965, Pages 21-28). Fabianek et al. show that the amount of chondroitin sulfate (non-HA GAGs) in rabbit skin is 319 μg/g dry mass with a 3:1 conversion factor to wet mass. Applying this correction the inventors find that the concentration of chondroitin sulfate (non-HA GAGs) is 106 ng/mg wet mass (Fabianek, Comparative Biochemistry and Physiology. Volume 14, Issue 1, January 1965, Pages 21-28). This value is 5.47 times smaller than that found in the control rabbit skin measured in this study. This difference could be explained by factors related to sample processing and differences in extraction and GAG determination techniques.

In in vitro skins, the amount of total GAGs, corresponding to the sum of sulfated GAGs (chondroitin sulfate) and HA is 145.8 ng/mg, compared to 883.9 ng/mg in the control rabbit skin measured in this study. There is 5.2 times less HA and 6.7 times less sulfated GAGs, respectively, than in the rabbit skins analyzed. Nevertheless, comparison of these results with those found in Armstrong and Bell and Fabianek et al. reveals a difference of minus 17% of sulfated GAGs and 9.5 times less HA between rabbit skin and in vitro skin.

A mass level of sulfated GAGs of 87.9±2.2 ng/mg as well as a HA level of 57.86±4.9 ng/mg were measured in in vitro skins. Although these values are lower than those measured in rabbit skins, the concentration of sulphated GAGs (chondroitin sulphate) is close to that found in the literature at 106 ng/mg for a rabbit skin.

### F. Characterization of the In-Vitro Leather

### 1. Differential Scanning Calorimetry (DSC)

A property commonly used to characterize collagen, whether native, structurally modified or chemically modified by tanning, is hydrothermal stability: this is defined as the effect of moist heat on the integrity of the material, usually in terms of denaturation transition (A. D. Covington, R. A. Hancock, I. A. Ioannidis, J. Soc. Leather Technologists Chemists 73, 1989) 1:8). The value of this parameter, depending on the processing (tanning), is an important physical parameter in the characterization of hides and their typical use. Covalent bonds, created especially during the tanning process by cross-linking of the tanning agents with the collagen fibers, increase the size of the cooperating units by inter- and intramolecular cross-linking and increase the denaturation temperature (Td) (S. Menashi, A. Finch, P. J. Gardner, D. A. Ledward, Biochem. Biophys. Acta 144 (1976) 623:625). Denaturation is defined as a transition from the triple helix to a randomly wound form, occurring in the domains between the cross-links (M. Komanowsky, J. Am. Leather Chemist Assoc. 86 (8) (1991) 269:28). The bonds that stabilize the superhelix are hydrogen bonds, hydrophobic bonds, van der Waal bonds and interactions between oppositely charged residues on the side chains. The non-random distribution of ionizable and hydrophobic side chains along the repeating unit results in the appearance of charged and hydrophobic patches that contribute to the stabilization of the structures through electrostatic and hydrophobic interactions (G. S. Young, Stud. Conserv. 43 (2) (1998) 65:79).

This increase in Td varies with the nature and type of reactive groups involved. Thus, the method of tanning the leather directly influences its shrinkability. Traditionally, the Td of leather tanned with vegetable agents oscillates around 70° C., while the Td of chrome tanned leather oscillates around 80-90° C. For leather tanned with synthetic tanning agents, the Td is around 75° C. (R. Komsa-Penkova, R. Koynova, G. Kostov, B. G. Tenchov, Biochem. Biophys. Acta 129 (1996) 171:181; C. Chahine, Changes in hydrothermal stability of leather and parchment with deterioration: a DSC study).

Hydrothermal stability of collagen in skin and leather is characterized by a shrinkage of the material when heated in water at a defined temperature. Differential scanning calorimetry (DSC) allows to study the enthalpy changes which are associated with the denaturation of collagen, of which shrinkage is the macroscopic manifestation. This work aims to analyze the hydrothermal stability of leather obtained according to the method of the invention. The unseeded membrane macroporous scaffold and the in-vitro skin were used as reference materials before tanning. Leather tanned of in-vitro skin according to the present invention with two different methods, aldehyde were tested to compare the effect of the tanning method over the temperature of denaturation (Td) of tanned leather. The results of the analysis and measurements have shown that the tanned leather of in-vitro skin has a hydrothermal stability close to that of a tanned leather of animal origin.

### 1.1 Differential Scanning Calorimetry (DSC) Protocol

Measurements were performed using a 2014 Polyma DSC calorimeter (Netzsch). It was calibrated for temperature and heat flux with indium (melting point: 156.6° C. and latent heat of fusion: 28.45 J/g). Samples were weighed (3 mg), immersed in water for several hours, and then hermetically sealed in aluminum crucibles. Thermal changes are measured relative to those observed on a reference sample, with purging performed via a nitrogen atmosphere. The two trays (reference and sample) are heated separately at a constant rate of 5° C./min ranging from 10 to 95° C. The instrument records the rate at which heat must be applied to maintain both tests at the same temperature. When the test sample undergoes an endothermic process, the energy input required to maintain the temperature increases and gives a signal on the rising temperature curve.

### 1.2 Results

Differential Scanning Calorimetry (DSC) was used to determine the denaturation temperature (Td) of the samples. This denaturation temperature is obtained via the analysis of the interval of variation of the transition temperature (onset and offset) with a peak assimilated to the denaturation temperature (Td). This last method is more adapted to the present complex system (scaffold and biomaterial) (Carsote, C., Badea, E. Herit Sci 7, 48 (2019)).

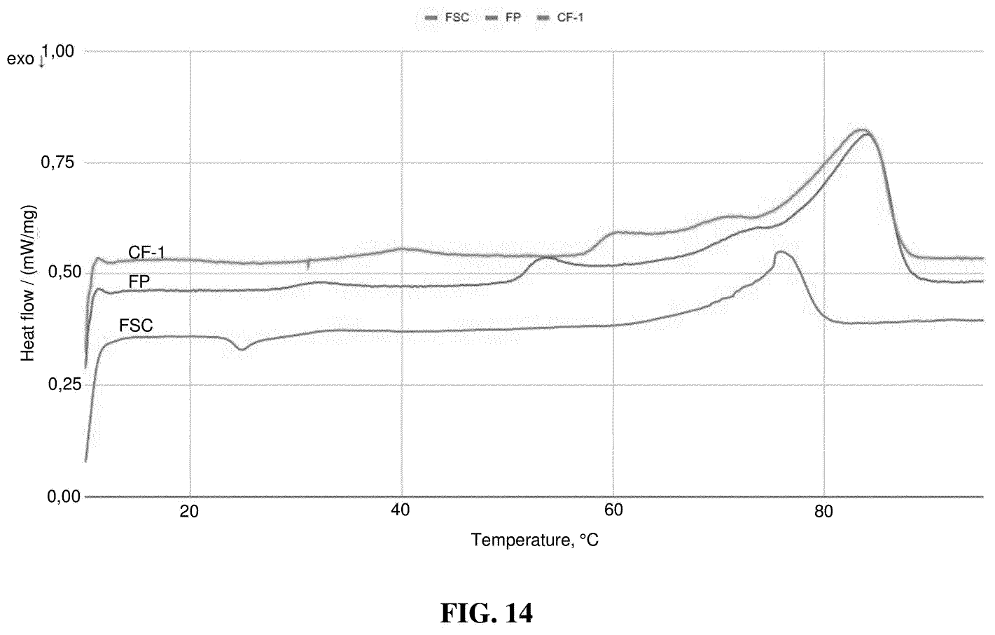

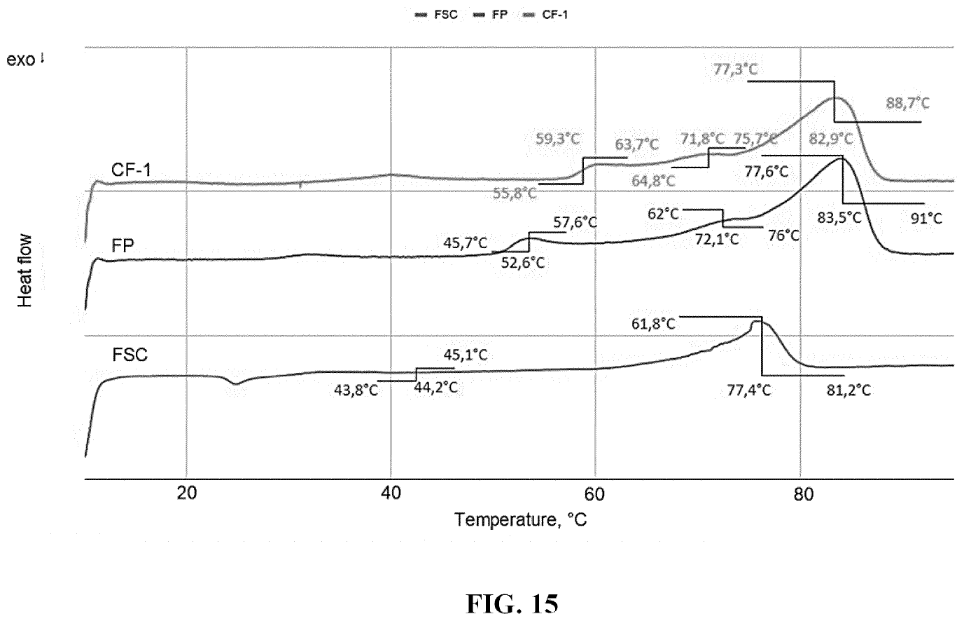

The measurements were performed using a Netzsch DSC 2014 Polyma Calorimeter at the constant speed of 5° C./min ranging from 10 to 95° C. FIG. 14 shows the DSC curves of different samples: macroporous scaffold (FSC), in-vitro skin (FP), leather tanned of in-vitro skin with aldehyde (CF-1). FIG. 15 shows the decomposition of the peaks and the determination of the denaturation temperatures of the different species present in the sample: Onset temperature (Tₒₙₛₑₜ) of the beginning of the transition, denaturation temperature corresponding to the peak (Td) and offset temperature of the end of the transition (T_{offset}).

Looking closely at the thermograms obtained for the different samples, these can be broken down into two (for the FSC sample) and three (for the FP, CF-1, and CF-2 samples) peaks/deconvolutions with intervals corresponding to different denaturation temperature occurring in this complex system. The denaturation temperatures for each sample and for each of these peaks/deconvolutions are presented in Table 13. The results show that the lowest recorded Td of 44.2° C. corresponds to the first peak/deconvolution of the unseeded macroporous scaffold (FSC) with a barely detectable enthalpy of denaturation. The first peak/deconvolution of the in-vitro skin (FP) increases to 52.6° C., this minority peak in terms of enthalpy could correspond to collagen not modified by tanning having a value close to that obtained by Carsote et al. of 54.2° C. for non-chemically modified collagen (Carsote, C., Badea, E. Herit Sci 7, 48 (2019)).

The Td of the second peak/deconvolution of the unseeded macroporous scaffold (FSC) rises to 77.4° C., this peak being the largest in terms of enthalpy should correspond to the collapse of the geometry of the unseeded scaffold at high temperature. For the second peak/deconvolution of the in-vitro skin (FP) hides and the aldehyde (CF-1) tanned leather, the denaturation temperatures are almost similar and are 72.1 and 71.8 respectively. This second peak/deconvolution having a higher enthalpy than the first one, should correspond to more or less chemically modified collagen fibers and biomaterial entangled in the scaffold forming a more thermally resistant composite material.

The highest denaturation temperatures that have been recorded for the in-vitro skin (FP) and aldehyde (CF-1) tanned leathers are 83.5 and 82.9 respectively. This third and last peak with the highest enthalpy would correspond to collagen fibers chemically bound by the tanning agent and the biomaterial which is more or less chemically modified and entangled in the scaffold, thus the whole forms a highly temperature resistant composite material. The presence of the collagen and the chemically modified biomaterial allows the composite material to resist thermal denaturation and to maintain the geometry of the composite at about 83° C. compared to 77.4° C. for the unseeded macroporous scaffold.

TABLE 13
Denaturation temperatures determined by DSC

| | Denaturation temperature (° C.) | | | | | | | | |
|---|---|---|---|---|---|---|---|---|---|
| | 1^{st} deconvolution | | | 2^{nd} deconvolution | | | 3^{rd} deconvolution | | |
| Sample | Tₒₙₛₑₜ | Td | T_{offset} | Tₒₙₛₑₜ | Td | T_{offset} | Tₒₙₛₑₜ | Td | T_{offset} |
| FSC | 43.8 | 44.2 | 45.1 | 61.8 | 77.4 | 81.2 | — | — | — |
| FP | 45.7 | 52.6 | 57.6 | 62.0 | 72.1 | 76.0 | 77.6 | 83.5 | 91.0 |
| CF-1 | 55.8 | 59.3 | 63.7 | 64.8 | 71.8 | 75.7 | 77.3 | 82.9 | 88.7 |

These results show enthalpy changes associated with collagen denaturation and the temperature at which the phenomenon occurs for leather tanned (FIG. 14 and Table 13). Covalent bonds, created especially during tanning, stabilize the collagen fiber bonds and increase their denaturation temperature (Td). This stabilization by tanning is observed in particular when increasing the Td of the first peak/deconvolution of the in-vitro skin (FP) from 52.6° C. before tanning, to 59.3° C. for the aldehyde-tanned leather (CF-1). Concerning the second and third peaks/deconvolutions, the inventors observed a dominant effect of the formation of the composite mixing collagen, biomaterial and scaffold compared to tanning on the denaturation temperatures (Td). Indeed, the Td of these second and third peaks/deconvolutions are similar between the in-vitro skin and the aldehyde (CF-1) tanned leathers.

These results show that the effect of tanning agents on the Td of animal hides can be found in in-vitro skin tanned leathers.

Larsen et al. measured the Td of different tanned hides including Mimosa and Sumach hides (Larsen, Rene & Vest, M. & Nielsen, K . . . (1993), Journal of the Society of Leather Technologists and Chemists. 77. 151-156). They compared the Td of an untanned hide to that of these two leathers. Their results showed a significant increase in Td after tanning, 75.3 and 79.3° C. respectively for Mimosa and Sumach versus 56.5° C. for the untanned hide. In addition, they reported a non-uniformity of measurements between the two leather samples and this was explained by an organization of the collagen fibers that occurs in a tanning-dependent manner.

As discussed before, the thermograms obtained for the different samples of the present invention are complex, as they are composed of two or three different peaks/deconvolutions. Similar analyses of the decompositions of the DSC thermograms have been reported in thermogravimetric studies for co-polymers or composite systems (Luo K, Wang L, Chen X, Zeng X, Zhou S, Zhang P, Li J. Polymers (Basel). 2020 Nov. 9; 12(11):2631; Athanasoulia, Ioanna-Georgia and Tarantili, Petroula A. Pure and Applied Chemistry, vol. 89, no. 1, 2017, pp. 141-152). The DSC curves corresponding to the composite systems showed fluctuations endothermic fluctuations located in the region of degradation and rearrangement of polymeric chains. This behavior allowed to define a denaturation region of the system by estimating an onset denaturation temperature (Tₒₙₛₑₜ), a peak temperature (Td) and an offset denaturation temperature (T_{offset}). These temperatures successively reflect the onset of denaturation which corresponds to the reorganization of chains of different natures, the optimum of deformation and the post-deformation stability of the chains. This phenomenon is similar to the observations obtained with samples of the present study.

In each thermogram of FP and FC-1, three fluctuations are clearly visible, bounded by pre- and post-denaturation stability. This behavior is not detectable in the case of the scaffold (FSC) which is, unlike the other samples, a non-complex system that does not contain biomaterial. In the case of cultured in-vitro skin (FP), the complex system consists of the macroporous scaffold (FSC) and the biomaterial that encompasses the cells with the collagen fiber-rich extracellular matrix (ECM). Tanned leathers (CF-1 and CF-2) constitute a complex ternary system encompassing the scaffold (FSC), the biomaterial especially the cross-linked collagen fibers and the tanning agent. Thus, as shown by the curve decomposition in FIG. 15, regions of denaturation corresponding to the state of the material are defined. From these decompositions, leathers of the invention show similar denaturation characteristics with denaturation temperature ranges that vary between 55.8 and 88.7° C. for the aldehyde tanning method. The average Td (72.2° C.) characteristic of invention leather remains very close to the range (76.6; 87° C.) of the newly aldehyde tanned leather (Bai, Xue & Jinming, Chang & Chen, Yi & Fan, Haojun & Shi, Bi. (2013)). Journal of the American Leather Chemists Association. 108. 404-410).

Another important factor that strongly influences the Td value is the water content of the material. Thus, the lower the water content, the higher the Td. This phenomenon is attributed to the fact that during drying, thermal stabilization is enhanced by the formation of strong inter- and intra-molecular and ionic bonds between the acid and basic groups. Classically, the phenomenon of shrinkage is measured in aqueous medium, and it is for this reason that the measurements of Td are carried out in excess of water (A. Finch, D. A. Ledward, Biochim. Biophys. Acta 278 (1972) 433-439).

In the present study, the inventors have kept these classical conditions in the realization of the measurements. Thus, considering these conditions, the analysis conducted by DSC could be very useful to evaluate the thermal stability of leather and allowed to compare between the two tanning methods adopted.

In order to obtain references between the measurements obtained by DSC with temperature resistance measurements commonly used in tanneries, the inventors used a shrinkage temperature tester (Tr) specially designed for leather. This device consists of a beaker filled with water in which leather specimens are immersed under mechanical tension. The leather samples are then subjected to a range of temperatures (20-100° C.) using a heating plate placed under the system. The shrinkage of the sample at a given temperature (Tr) is recorded by the displacement measured by a centesimal dial gauge (Borletti). The shrinkage temperatures (Tr) of in-vitro skin (FP), aldehyde-tanned leather (CF-1) were measured and presented in Table 14. The results show that the Tr of the in-vitro skin (FP) oscillates around 65° C. which corresponds to a temperature in the interval between the first and second peak/deconvolution obtained by DSC (FIG. 14, Table 13, FP). On the other hand, the Tr of aldehyde-tanned leather oscillates around 75° C. which corresponds to a temperature located at the interval between the second and third peak/deconvolution obtained by DSC (FIG. 14, Table 13, CF-1).

TABLE 14
Shrinkage temperature (Tr) of in-vitro skin
and tanned leather of in-vitro skin.

| Sample | Shrinkage temperature (Tr) |
|---|---|
| FP | 65° C. |
| CF-1 | 75° C. |

Shrinkage temperature (Tr) measurements obtained using a tester are less accurate (thin sample thickness and displacement measurement made by an observer) compared to measurements made by DSC. Nevertheless, this method is more commonly used in tanneries and provides a useful framework for comparison between measurements made by DSC and measurements made in tanners and tradesmen (G. S. Young, Stud. Conserv. 43 (2) (1998) 65:79). The results showed that aldehyde-tanned leather of the invention has a thermal stability very close to a newly tanned animal leather with the same method.

### 2. Mercury Intrusion Porosimetry

### 2.1 Materials and Methods

Mercury porosity measurements were performed with a micrometer porosimeter (AutoPore IV 9500, Micrometrics) with a maximum applied pressure of 413 MPa. The samples used for this measurement were: scaffold (FSC) and aldehyde tanned leather of in vitro skin (CF-1).

The measurements were performed at 24° C. The FSC samples were pre-dried at 50° C. for 24 h and the CF-1 samples were pre-dried at 80° C. for 24 h. The equilibration time for each pressure point was set to 10 s. Blank correction was used before measurement. The sample was cut into 3 cm×2 cm pieces (about 0.6 g) and was first degassed and then filled with low-pressure distilled mercury. The mercury-filled penetrometer was removed from the low-pressure port, weighed, and then placed in the high-pressure port. Mercury was introduced into the pores of the samples with the increase in pressure. The recorded pressure and volume of mercury introduced into the pores were used to calculate the porosity.

### 2.2 Results

### Porosimetric Properties of In-Vitro Skin Leather

The total porosity (8) that is calculated from the MIP test have been listed in Table 15.

TABLE 15
Total porosities of scaffold (FSC), in vitro skin (FP),
and aldehyde-tanned leather (CF-1) compared to untanned
bovine skin and chromium-tanned sheep leather.

| | ε (%) |
|---|---|
| FSC | 87.21 |
| FP | 14.78 |
| CF-1 | 51.63 |
| Untanned bovine skin | 59.07 |
| Sheep leather | 57.26 |

According to the data extracted from the MIP analyses shown in Table 15, the scaffold (FSC) has a high percentage of total porosity (¿) (87.21%). The design of the structural characteristics of the scaffold according to the present disclosure show its high degree of porosity with an average pore diameter compatible with the seeding and penetration of animal cells with an average diameter of 20 μm (Al Tawil, E., et al. (2018). European Polymer Journal).

The total porosity decreases sharply in in vitro skin (FP) and reach 14.78%. This decrease is explained by the occupation of the pore volume by biomatter: cells and components of the extracellular matrix (ECM) secreted by the cells. This biomatter is the essential constituent of the neoformed tissue of in-vitro skins, and which is found in tanned hides as in conventional animal leather (He, Xiu et al. (2019). Journal-American Leather Chemists Association. 114. 41-47).

Tanning is a step that transforms skin, either conventional or in vitro, into leather by creating chemical bridges between the collagen fibers of the ECM. As a result of this network cross-linking, spaces called macropores, mesopores and nanopores are created between the collagen chains. This porosity of the leather is an appreciated property and is the reason for its breathing phenomenon. The leather tanned with aldehyde (CF-1) show a total porosity reduced to 51.63%. These results are very comparable to those obtained for chrome-tanned sheep leather (Table 15).

### 2.3 Discussion

The porosimetric properties of a material is one of its essential characteristics, they are parameters of choice having an influence on their use framework and in the types of products using the material. In the case of leather, the porosimetry of the different types of leather guides its end use: decoration, clothing, leather goods, furnishings, among others. It also serves as an indicator of changes made by its manufacturing process, especially during the retanning and finishing phases. In this report, mercury intrusion porosimetry (MIP) was used to measure the porosimetric properties of leathers obtained from in-vivo skin of the present disclosure. The results showed that the scaffold has adequate porosity for the culture of animal cells. The growth of cells within the scaffold allowed for the colonization of the scaffold by the cells and the deposition of biomatter, an essential component of the in vitro skins. Leathers of in-vivo skin present a total porosity comparable to that obtained for conventional animal leathers.

### 3. Water Vapor Permeability

### 3.1 Material and Methods

### Material

Water permeability measurements were performed using the permea-diffusiometer (IGAsorp, Hiden Isochema). It is composed of two compartments, one upstream and one downstream of the sample. During the measurement, the permeant (water molecules) is introduced into the upstream compartment, while the downstream compartment is swept by a dry gas (N2 BIP, Air Products). Hydrometric detection was used. During the measurement, the water-enriched gas is measured with a mirror hygrometer, probe 1311 DR from General Eastern, (Elcowa-Mulhouse).

The experimental setup used for the measurements is composed of:
A permeation cell, where the sample to be tested is located, placed in an enclosure thermostated at 23° C. and consisting of two compartments, one in stainless steel and the other in Plexiglas;
A feed upstream of the sample where the purge gas, technical grade nitrogen, circulates in a first step, then the permeant in a second step;
A feeder downstream of the film where BIP type nitrogen (gas) circulates, serving as a carrier gas to the mirror hygrometer.

Measurements were performed at 23° C. with 50% relative humidity. The sample used for this measurement is aldehyde tanned leather (CF-1). The active surface area of the sample is 3.6 cm²

### Experimental Setup

The differential permeation measurement is performed in two successive steps. The first step, called the purge step, will remove as much water and residual molecules as possible from the sample, the cell and the gas circuit. The nitrogen then sweeps the upstream and downstream compartments until a stable and low water content is obtained. In the second step, which is the measurement step, water vapor at 50% relative humidity is introduced into the cell. The permeant will then diffuse through the sample and will be carried by the carrier gas downstream of the sample to the detector (mirror hygrometer). The evolution of the flow is recorded in real time from the direct measurement of the variation of the dew point temperature.

### Calculation of Flux and Permeation

The receiving compartment, supplied with dry nitrogen (vapor pressure pᵢₙ) (BIP nitrogen) at a flow rate f=9.3 cm³/s and a total pressure of pt=1 atm, is not completely free of water and its vapor pressure increases from pᵢₙ to pₒᵤₜ (vapor pressure at the compartment inlet and outlet). The flux J(L,t) is given by:

$$J ( L , t ) = ( f \cdot ( pout - pin ) / S \cdot R \cdot Tr \qquad Eq . 1$$

with R the constant of perfect gases, S the exposed surface and Tr the temperature (K) at which the measurement is made.

The permeability coefficient P is determined from:

$$P = Jst \cdot L / Δ a \qquad Eq . 2$$

with Jₛₜ, the stationary flux, L is the thickness of the sample and Δis the activity difference on both sides of the sample.

### 3.2 Results

Water vapor permeability measurements were made using a permea-diffusiometer at 23° C. and 50% relative humidity. The measurements recorded in real time are shown in Table 16. In vitro skin leather tanned with aldehyde (CF-1) have permeability coefficients of 3.47 mg/cm²·h. Representative values for bovine skins have also been added to this table (Cattle leather: Radwag wagi elektroniks—testing laboratory. https://radwag.com/pl/).

TABLE 16
Water vapor permeability of the different samples recorded
at 23° C. and in the presence of 50% relative humidity.

| | | Water vapor permeability |
|---|---|---|
| Samples | Thickness (μm) | (mg/cm² · h) |
| CF-1 | 455.2 ± 0.7 | 3.47 |
| Cattle leather | 230 | 0.7-5.3 |

### 3.3 Discussion

There are different methods to measure the water vapor permeability of leather, the most widely used is the measurement according to ISO 14268:2013 (Leather-Physical and mechanical tests-Determination of water vapor permeability (ISO/FDIS 14268:2012). 2013). Water vapor permeability depends on a number of operations during the leather manufacturing process, including tanning and finishing. Leather is manufactured in the laboratory by combining scaffolding and animal cells.

Leathers (CF-1) were produced using aldehyde tanning. Water vapor permeation measurements recorded 3.47 mg/cm²·h for CF-1. It is known that leather is generally considered to have good water vapor permeability (WVP) has a permeability rate above 20 and a water vapor permeability value above 0.8 (Leather international. 18 Sep. 2001). The present results show that leather has good water vapor permeability consistent with that found in conventional leathers. Water vapor permeability analyses of different types of animal leathers have been performed (Skenderi Z., et al. Ergonomics 2013 Jun. 12-15, 2013, Zadar, 9-14; Smiechowski K., et al. Journal of the Society of Leather Technologists and Chemists, ISSN 0144-0322, Vol. 98, Num. 6, 2014; Radwag wagi elektroniks-testing laboratory. https://radwag.com/pl/). Smieshoweski et al. measured the water vapor permeability of different hides using a standard method and a method developed in their laboratory. The different types of leather selected for their study showed a water vapor permeability varying between 0.3 and 4.9 mg/cm²·h for samples with thicknesses around 1.2 mm. On the other hand, a study published by the Radwag Testing Laboratory shows that bovine leather used in the footwear industry has a water vapor permeability that varies between 0.7 and 5.3 mg/cm²·h depending on the method of manufacture: creased, pressed or untreated.

Based on these results, the inventors observed that in-vitro skin leather has a water vapor permeability comparable to that of conventional leathers. The values recorded are very closed to those published on measurements made on animal origin leather samples. It is important to note that the present tests were performed on unfinished tanned leathers with an average thickness of 0.45 mm; it was shown that water vapor permeability increases significantly as the thickness of the leather decreases (Smiechowski K., et al. Journal of the Society of Leather Technologists and Chemists, ISSN 0144-0322, Vol. 98, Num. 6, 2014).

Considering these results, measurements of the water vapor permeability of in-vitro skin leathers differing in thickness, tanning methods and potential finishes will allow us to find the manufacturing factors important for varying the water vapor permeability of in-vitro skin leathers.

1. A method for producing leather comprising the steps of:
i) culturing fibroblasts in vitro on a honeycomb macroporous polymeric scaffold to obtain a tissue,
wherein a surface of said scaffold comprises macropores of diameter comprised between 100 and 280 μm, with a surface pore area distribution comprised between 40% to 95% and pore wall thickness below 70 μm; and
ii) tanning said tissue thereby forming said leather.

2. The method of claim 1 wherein said scaffold comprises polyester.

3. The method of claim 1 wherein said scaffold comprises a polyester at a concentration comprised between 1 and 50% (w/w).

4. The method of claim 1 wherein said macropores are interconnected with micropores.

5. The method of claim 1 wherein said scaffold has a tensile strength comprised between 0.1 and 10 MPa.

6. The method of claim 1 wherein said scaffold has a Young's modulus comprised between 5 and 1 MPa.

7. The method according to claim 1 wherein said scaffold is obtained by non-solvent induced phase separation processes (NIPS).

8. The method according to claim 1 wherein said scaffold is an asymmetric scaffold comprising macropores that are homogeneously but not regularly distributed over the whole thickness of the scaffold, in particular which presents a first face comprising macropores opened at the surface of diameter comprised between 100 and 280 μm, with a surface pore area distribution comprised between 40% to 95%, and pore wall thickness below 70 μm and a second opposite face comprising only nanopores of diameter below 10 nm.

9. The method according to claim 1 wherein said scaffold is a symmetric scaffold comprising macropores which presents two faces comprising macropores of diameter comprised between 100 and 280 μm, with a surface pore area distribution comprised between 40% to 95%, and pore wall thickness below 70 μm.

10. The method according to claim wherein a bioactive molecule is grafted at the surface of said scaffold.

11. The method of claim 10 wherein said bioactive molecule is collagen, glucide, or glucide derived molecules such as glycosaminoglycan.

12. A tissue obtainable after the step a) of the method according to claim 1 comprising fibroblastic cells cultured on a macroporous scaffold as defined in claim 1.

13. The tissue according to claim 12 having at least two denaturation temperatures (Td) determined by differential scanning calorimetry (DSC) assay.

14. The tissue according to claim 12 wherein said tissue is isotropic at the surface of said tissue.

15. The tissue according to claim 12 comprising a percentage of total fatty acid mass in the tissue total dry mass of less than 1% (w/w).

16. A tanned leather obtainable by the method according to claim 1.

17. The tanned leather according to claim 16 having at least two denaturation temperatures (Td) determined by differential scanning calorimetry (DSC) assay.

18. The tanned leather according to claim 16 having a total porosity between 30 and 60%.

19. (canceled)

20. The method according to claim 1 wherein said scaffold comprises biodegradable or biosourced polyester.

21. The method according to claim 4 wherein said micropores have a diameter of less than 20 μm.
